(19) United States
(12) Patent Application Publication
(10) Pub. No.: US 2025/0375571 A1
(43) Pub. Date: Dec. 11, 2025

(54) METHODS AND DEVICES FOR REMOVING AGGLOMERATIONS FROM OF A VISCOUS MATERIAL

(71) Applicant: SPIDERWORT BIOTECHNOLOGIES INC., Ottawa (CA)

(72) Inventors: Andrew E. Pelling, Gloucester (CA); Ryan Hickey, Ottawa (CA); Joseph Christopher Samuel ODDING, Gatineau (CA); Allison TRAN, Ottawa (CA)

(21) Appl. No.: 18/876,264

(22) PCT Filed: Oct. 27, 2023
(86) PCT No.: PCT/CA2023/051437

§ 371 Date: Dec. 18, 2024

Related U.S. Application Data
(60) Provisional application No. 63/419,886, filed on Oct. 27, 2022.

(51) Int. Cl. A61M 5/165; A61M 5/31

(52) U.S. Cl. CPC A61M 5/165; A61M 5/3145; A61M 2205/7545; A61M 2210/04

(57) ABSTRACT

A method of filtering or removing agglomerations of individual particles from a viscous material such as a dermal filler formulation is provided. The method involves filling the viscous material in an extrusion system comprising a sieve or filter placed inside, outside, adjacent to or outside the system. The material is forced to pass through the filter/sieve which causes filtration/removal of the agglomerates present in the viscous material owing to its larger size. Alternatively, the pressure-based filtration cause separation of the individual particles causing breakdown of the agglomerates, thereby allowing the viscous material to pass through the extrusion system without any occlusion problems. The filtered viscous material is suitable for administration to the patients. The invention also describes devices to carry out the proposed pressure-filtration technique.

### FIELD OF INVENTION

The present invention relates generally to filtering agglomerations from viscous injectable materials. More specifically, the present invention relates to methods and devices for removing agglomerations from viscous injectable materials such as dermal fillers.

### BACKGROUND

Certain drug formulations could be highly viscous in nature which could pose an issue when they are administered as injectables. A problem that naturally arises from this densification is the formation of aggregates. These agglomerates create non-uniform zones within the hydrogel, sol, or colloid which lead to a lack of control over the final injectable material. Often times rheological properties of the viscous material are altered which results in poor performance in the syringe upon administration.

Agglomerations in viscous materials potentially lead to two principal issues: occlusions and uneven extrusion forces. Importantly, clumps of individual particles can block the needle and prevent proper delivery of the medical formulation. Likewise, partial blockages may require excessive forces, which when dislodged, can lead to the delivery or injection of a larger amount of material than is desired. This could severely impact the end result, as the material isn't accurately delivered as desired.

Furthermore, there are several secondary issues associated with uneven distribution of particle sizes. For example, a non-uniform material would have different mechanical properties which could trigger different immune and host cell responses in the body. For instance, a more uniform material can conceivably have a more uniform extracellular matrix deposition than clumps of individual particles that restrict the surface area for matrix deposition to occur.

One example of such viscous material is dermal fillers. Certain viscous materials that are prepared using powders could be diluted or reconstituted to adjust viscosity, however, diluting or reconstituting does not work for viscous materials, as the problem reoccurs when the material is thickened. For some viscous materials such as dermal fillers as described by the Applicant in international application WO2021/248,236 it is desirable to adjust the viscosity of the filler material. For example, a low viscosity may allow for easier injection, whereas a high viscosity may maintain uniform particle arrangement and/or may maintain volume for longer periods of time, for example. Therefore, during production of dermal fillers, the particles produced must be concentrated to achieve a desired viscosity. In order for this material to be used as an injectable, it must be able to pass through needles or cannulas for effective delivery. Furthermore, certain applications may require certain viscous materials to have specific particle sizes or a substantially uniform or controlled distribution of particle sizes. A common solution is to use large bore needles or cannulas, however, large needles are painful and inconvenient for patients. Another common approach to overcome problems associated with agglomerations is to size, and sort individual particles, however, it may result in final materials with different mechanical properties which is not desirable.

Accordingly, methods and/or devices are desired that could help in overcoming the shortcomings noted above.

### SUMMARY OF INVENTION

Methods of removing agglomerations of individual particles from viscous materials are provided herein. The methods can also be used to filter and size the individual particles of viscous materials to achieve uniform mixing of components and a substantially uniform matrix for administration of the viscous material. In one embodiment the method includes removing or filtering agglomerations of individual particles from a viscous material by filling the material in a extrusion system or a first injection device comprising at least one filter/sieve. In some embodiments, the extrusion system may comprise a series of filters/sieves. The viscous material is then passed through the filter/sieve placed inside, outside, adjacent to or outside the extrusion system or the first injection device, which causes removal or filtration of the agglomerations, thereby making it suitable for administration to patients. The passing step may additionally require forcing the material through the filter/sieve under pressure. These methods are suitable for removing/filtering agglomerations of individual particles from a broad variety of viscous materials including dermal fillers.

In some instances, the method may be executed differently, wherein the viscous material is first filled or collected in a first collection vessel. The material is then passed through at least one filter/sieve fitted inside the first collection vessel, and the filtered material is collected in a second collection material, where the material is stored before it's administered to the patients. The filtered material stored in the second collection vessel can be transferred to individual administration/transfer syringes prior to direct administration to the patients. Use of the methods for removing/filtering agglomerations and for other purposes are described as well.

Devices for removing agglomerations are provided as well. A device for removing agglomeration may comprise a extrusion system or a first injection device with at least one filter/sieve fitted inside the extrusion system. The extrusion system may optionally be connected to a loading vessel/transfer syringe or a second injection device, which could be employed to store the filtered viscous material or for direct administration of the material to patient(s). A different version of the device is provided as well, where the material is collected in a first collection vessel that has the filter/sieve within its body. In some embodiments, the first collection vessel may comprise the extrusion system as defined hereinbefore. The first collection vessel may optionally be connected to a second collection vessel, which allows the filtered material to be stored therein, before the material is administered to patient(s).

### BRIEF DESCRIPTION OF DRAWINGS

FIG. 1 shows results of needle occlusion test with mercerized viscous material.

FIG. 1(A) shows a 27 G needle and syringe used for extrusion. FIG. 1(B) shows extrusion of mercerized viscous material. FIG. 1(C) provides an example of 3D printing or controlled injection/extrusion.

FIG. 2 shows results of sieving test where individual particles/agglomerates that were too large to pass through the sieve are collected in a separate container.

FIG. 3 show results of slip-plane modification test wherein extrusion force profiles for the 0% gelatin formulation (red), and 5% gelatin formulation (blue) are shown. An extrusion rate of 1 mm/s was used. The material was extruded through 27 G needles from 1 cc syringes.

FIG. 4 shows a mesh employed during pressure drive sieving protocol.

FIG. 5 shows a microscope image of the mesh (with 10× magnification) employed during pressure-drive sieving.

FIG. 6 shows particle-size and morphology results which were investigated/checked after pressure-drive sieving. The particles were stained using Congo Red, imaged at 10× on the SZ16 Stereomicroscope (Scale=500 μm).

FIG. 7 shows the particle size distribution of the viscous injectable material that passed through the mesh in pressure-drive sieving process.

FIG. 8 shows in-line pressure filtration apparatus of the present invention.

FIG. 9 shows in-syringe pressure filtration apparatus of the present invention where the mesh is placed at the base of the inner portion of the syringe.

FIG. 10 shows in-line syringe pressure filtration apparatus where the mesh is housed in a custom luer lock connector which is connected between the syringe and the needle.

FIG. 11 shows a block diagram of the filtration process.

FIG. 12 shows a block diagram of the small, medium and large scale filtration process.

### DETAILED DESCRIPTION

The following description is of preferred embodiments by way of example only and without limitation to the combination of features necessary for carrying the invention into effect.

All terms are intended to be understood as they would be understood by a person skilled in the art. Unless defined otherwise, all technical and scientific terms used herein have the same meaning as commonly understood by one of ordinary skill in the art to which the disclosure pertains. The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application. Accordingly, the terminology used herein is for the purpose of describing particular embodiments only, and is not intended to be limiting.

The terms “first injection device”, “extrusion system”, “and “first collection vessel” refer to a container where the material to be de-agglomerated or filtered is collected prior to the filtration step.

The terms “second injection device”, “loading vessel”, “transfer syringe” or second collection vessel” refers to a container where the filtered or de-agglomerated material is stored after the filtration step or prior to administration to the patients.

To overcome the issues/problems associated with prior art techniques, the present inventors have attempted to identify a solution that would limit the number or occlusions or completely eliminate occlusion events. Apart from removing agglomerations present in the viscous material, the claimed technique sizes individual particles of the viscous material without affecting their characteristic features and/or morphology. It is pertinent to note that along with prevention of potential occlusion events, having a substantially uniform or controlled extrusion profile is extremely important for accurate delivery of drug formulations. To prevent pressure build-ups and uneven extrusion pressures, Applicant experimented with various commonly known techniques and devices to see if any prior techniques or devices could be employed to eliminate the problems associated with injecting viscous materials. It is relevant to note that the inventors identified and defined an occlusion event as a needle blockage or blockage of the extrusion system or discharge system, wherein the material ceased to flow through the needle i.e. clogged the extrusion system/discharge system. The various embodiments of invention are described in detail below.

Provided herein is a method of removing agglomerations of individual particles from a viscous material for administration to a patient. The method comprises filling the viscous material in an extrusion system comprising at least one filter/sieve, and passing the viscous material through the at least one filter/sieve to cause removal of the agglomerations of individual particles from the viscous material. The filtered viscous material produced by the method is suitable for administration to the patient.

In certain embodiments, the method does not change the characteristic features or morphology of at least 98%, at least 95%, at least 92%, at least 89%, at least 86%, at least 83%, or at least 80% of the individual particles of the viscous material.

In further embodiments, the passing step additionally comprises forcing the viscous material through the at least one filter/sieve under pressure/by application of pressure. In certain embodiments, the viscous material is forced through the at least one filter/sieve by application of pressure using a plunger, an extruder, a manual compression, a syringe pump, a platen compression, a roller for flexible tubes, a compressor, or a device capable of employing similar mechanism.

In some embodiments, the agglomerations are removed from the viscous material by breaking-up/disintegrating the agglomerations. The agglomerations are removed from the viscous material by filtering out/sieving out the agglomerations that are unable to pass through the at least one filter/sieve.

In some further embodiments, the viscous material is pre-filtered through a plurality of pre-filtering devices, wherein the pre-filtration step occurs prior to the passing step. In some embodiments, the viscous material is pre-filtered through a first pre-filtering device followed by a second pre-filtering device. In some alternate embodiments, the plurality of pre-filtering devices are a series of injection devices, injection syringes, discharge systems, injection devices with luer-lock connectors, compressible tubes, non-compressible tubes, cylinders, or large-scale syringes.

In some embodiments, the first and second pre-filtering devices are a series of injection devices, injection syringes, or large-scale syringes, wherein the first pre-filtering device has an aperture size greater than the second pre-filtering device. The aperture size of the first and the second pre-filtering devices may be in the range of 18G-34G.

In some embodiments, the viscous material is passed through the at least one filter/sieve having a pore size/an aperture size smaller than the individual particle size of the viscous material, and the pore size of the at least one filter/sieve may be in the range of 1-1000 μm and the individual particle size may be in the range of 20-1000 μm. In some alternate embodiments, the pore size of the at least one filter/sieve may be in the range of 25-500 μm and the individual particle size may be in the range of 40-500 μm. In some other embodiment, the pore size of the at least one filter/sieve may be in the range of 50-200 μm and the individual particle size may be in the range of 75-200 μm.

In some embodiments, the filtered viscous material is collected in a loading vessel/transfer syringe where the filtered viscous material does not occlude the loading vessel/transfer syringe when injected out of the loading vessel/transfer syringe. In some embodiments, the filtered viscous material achieves a substantially uniform or controlled extrusion profile when injected out of the loading vessel/transfer syringe and injects out of the loading vessel/transfer syringe without any pressure build-ups after the initial characteristic burst/break force.

In certain embodiments, the individual particles of the viscous material have an undamaged morphology. The filtered viscous material triggers an expected immune response in the patient upon administration. The extrusion system is may be an injection syringe, a discharge system, a luer-lock connector device, or a compressible tube. In some embodiments, the extrusion system may be a syringe, where the syringe has a needle size in the range of 18G-34G.

In some embodiments, where the loading vessel/transfer syringe is an injection syringe, a discharge system, a luer-lock connector device, or a compressible tube. In some embodiments, the loading vessel/transfer syringe may be a syringe, where the syringe has a needle size of 18G-34G. In some of the previous embodiments, volume of the extrusion system is greater than the volume of the loading vessel/transfer syringe, or where the extrusion system has a lower cross section compared to the loading vessel/transfer syringe.

In some of the previous embodiments, at least one filter/sieve may be a nylon mesh, stainless steel mesh, polytetrafluoroethylene mesh or nitrocellulose mesh. The at least one filter/sieve may be placed inside, outside, adjacent to, or screwed on to the extrusion system and could be replaced with an inline filter, a gated impeller, a static mixer, a high shear mixer, a viscous mixer or a sieving channel.

It is pertinent to note that the passing step sizes the individual particles of the viscous material to create a substantially uniform or controlled particulate matrix for administration. In some embodiments 1, wherein the extrusion system is a luer-lock connector device, wherein the at least one filter/sieve is fitted/disposed within the luer-lock connector portion of the device.

The viscous material is forced through the at least one filter/sieve fitted/disposed within the luer-lock connector portion under pressure/by application of pressure. The pressure could be applied using a plunger, an extruder, a manual compression, a syringe pump, a platen compression, a roller for flexible tubes, a compressor or a device capable of employing similar mechanism. In some embodiments, the pressure is applied using a plunger or an extruder.

In certain embodiments, the passing step may be repeated with a plurality of filters/sieves. In some embodiments, wherein the plurality of filters/sieves are of the same size or have varying sizes. In some embodiments, the pore size of the at least one filter/sieve is in the range of 1-1000 μm.

In some of the previous embodiments, passing the material through a plurality of filter/sieves reduces the viscosity or adjusts the particle size distribution of the viscous material to a desired level, preferably in the range of 50-750 μm. The proposed method may also reduce the extrusion force required for injecting out the viscous material from the extrusion system.

In certain embodiments, the viscous material is dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

Provided also is a method of removing agglomerations of individual particles from a viscous dermal filler material for administration to a patient that involves filling the dermal filler material in an extrusion system comprising at least one filter/sieve and passing the dermal filler material through the al least one filter/sieve causing removal of the agglomerations of individual particles from the dermal filler material. The filtered dermal filler material is suitable for administration to the patient.

In certain embodiments, the method does not change the characteristic features or morphology of at least 98%, at least 95%, at least 92%, at least 89%, at least 86%, at least 83%, or at least 80% of the individual particles of the dermal filler material.

In further embodiments, the passing step additionally comprises forcing the dermal filler material through the at least one filter/sieve under pressure/by application of pressure.

In certain embodiments, the dermal filler material is forced through the at least one filter/sieve by application of pressure using a plunger, an extruder, a manual compression, a syringe pump, a platen compression, a roller for flexible tubes, a compressor, or a device capable of employing similar mechanism.

In some embodiments, the agglomerations are removed from the dermal filler material by breaking-up/disintegrating the agglomerations. The agglomerations are removed from the dermal filler material by filtering out/sieving out the agglomerations that are unable to pass through the at least one filter/sieve.

In some further embodiments, the dermal filler material is pre-filtered through a plurality of pre-filtering devices, wherein the pre-filtration step occurs prior to the passing step. In some embodiments, the dermal filler material is pre-filtered through a first pre-filtering device followed by a second pre-filtering device. In some alternate embodiments, the plurality of pre-filtering devices are a series of injection devices, injection syringes, discharge systems, injection devices with luer-lock connectors, compressible tubes, non-compressible tubes, cylinders, or large-scale syringes.

In some embodiments, the first and second pre-filtering devices are a series of injection devices, injection syringes, or large-scale syringes, wherein the first pre-filtering device has an aperture size greater than the second pre-filtering device. The aperture size of the first and the second pre-filtering devices may be in the range of 18G-34G.

In some embodiments, the dermal filler material is passed through the at least one filter/sieve having a pore size/an aperture size smaller than the individual particle size of the dermal filler material, and the pore size of the at least one filter/sieve may be in the range of 1-1000 μm and the individual particle size may be in the range of 20-1000 μm. In some alternate embodiments, the pore size of the at least one filter/sieve may be in the range of 25-500 μm and the individual particle size may be in the range of 40-500 μm. In some other embodiment, the pore size of the at least one filter/sieve may be in the range of 50-200 μm and the individual particle size may be in the range of 75-200 μm.

In some embodiments, the filtered dermal filler material is collected in a loading vessel/transfer syringe where the filtered dermal filler material does not occlude the loading vessel/transfer syringe when injected out of the loading vessel/transfer syringe. In some embodiments, the filtered dermal filler material achieves a substantially uniform or controlled extrusion profile when injected out of the loading vessel/transfer syringe and injects out of the loading vessel/transfer syringe without any pressure build-ups after the initial characteristic burst/break force.

In certain embodiments, the individual particles of the dermal filler material have an undamaged morphology. The filtered dermal filler material triggers an expected immune response in the patient upon administration. The extrusion system is may be an injection syringe, a discharge system, a luer-lock connector device, or a compressible tube. In some embodiments, the extrusion system may be a syringe, where the syringe has a needle size in the range of 18G-34G.

In some embodiments, where the loading vessel/transfer syringe is an injection syringe, a discharge system, a luer-lock connector device, or a compressible tube. In some embodiments, the loading vessel/transfer syringe may be a syringe, where the syringe has a needle size of 18G-34G. In some of the previous embodiments, volume of the extrusion system is greater than the volume of the loading vessel/transfer syringe, or where the extrusion system has a lower cross section compared to the loading vessel/transfer syringe.

In some of the previous embodiments, at least one filter/sieve may be a nylon mesh, stainless steel mesh, polytetrafluoroethylene mesh or nitrocellulose mesh. The at least one filter/sieve may be placed inside, outside, adjacent to, or screwed on to the extrusion system and could be replaced with an inline filter, a gated impeller, a static mixer, a high shear mixer, a viscous mixer or a sieving channel.

It is pertinent to note that the passing step sizes the individual particles of the dermal filler material to create a substantially uniform or controlled particulate matrix for administration. In some embodiments 1, wherein the extrusion system is a luer-lock connector device, wherein the at least one filter/sieve is fitted/disposed within the luer-lock connector portion of the device.

The dermal filler material is forced through the at least one filter/sieve fitted/disposed within the luer-lock connector portion under pressure/by application of pressure. The pressure could be applied using a plunger, an extruder, a manual compression, a syringe pump, a platen compression, a roller for flexible tubes, a compressor or a device capable of employing similar mechanism. In some embodiments, the pressure is applied using a plunger or an extruder.

In certain embodiments, the passing step may be repeated with a plurality of filters/sieves. In some embodiments, wherein the plurality of filters/sieves are of the same size or have varying sizes. In some embodiments, the pore size of the at least one filter/sieve is in the range of 1-1000 μm.

In some of the previous embodiments, passing the material through a plurality of filter/sieves reduces the viscosity or adjusts the particle size distribution of the dermal filler material to a desired level, preferably in the range of 50-750 μm. The proposed method may also reduce the extrusion force required for injecting out the dermal filler material from the extrusion system.

In certain embodiments, the dermal filler material is dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

Provided is a method of removing agglomerations of individual particles from a viscous material for administration to a patient that involves filling the viscous material in a first collection vessel comprising at least one filter/sieve, passing the viscous material through the at least one filter/sieve causing removal of the agglomerations of individual particles from the viscous material and collecting the viscous material in a second collection vessel. The filtered viscous material collected in the second collection vessel is suitable for administration to the patient.

The first collection vessel comprises an extrusion system, wherein the extrusion system comprises the at least one filter/sieve. In some embodiments, the method does not change the characteristic features or morphology of at least 98%, at least 95%, at least 92%, at least 89%, at least 86%, at least 83%, or at least 80% of the individual particles of the viscous material.

In certain embodiments, the passing step additionally comprises forcing the viscous material through the at least one filter/sieve under pressure/by application of pressure. The viscous material may be forced through the at least one filter/sieve by application of pressure using a plunger, an extruder, a manual compression, a syringe pump, a platen compression, a roller for flexible tubes, a compressor or a device capable of employing similar mechanism.

In some embodiments, the agglomerations are removed from the viscous material by breaking-up/disintegrating the agglomerations. The agglomerations are removed from the viscous material by filtering out/sieving out the agglomerations that are unable to pass through the at least one filter/sieve.

In some alternate embodiments, the viscous material is pre-filtered through a plurality of pre-filtering devices, wherein the pre-filtration step occurs prior to the passing step. In some further embodiments, the viscous material is pre-filtered through a first pre-filtering device followed by a second pre-filtering device. The plurality of pre-filtering devices may be selected from a series of injection devices, injection syringes, discharge systems, injection devices with luer-lock connectors, compressible tubes, non-compressible tubes, cylinder, or large-scale syringes. The plurality of pre-filtering devices could be a series of injection devices, injection syringes, or large-scale syringes, where the first pre-filtering device has an aperture size greater than the second pre-filtering device.

In some embodiments, the aperture size of the first pre-filtering device may be selected from 18G-34G. In some further embodiments, the aperture size of the second pre-filtering device is selected from 18G-34G.

In certain embodiments, the viscous material is passed through the at least one filter/sieve having a pore size/an aperture size smaller than the individual particle size of the viscous material. In some embodiments, pore size of the at least one filter/sieve could be in the range of 1-1000 μm and the individual particle size may be in the range of 20-1000 μm. In some embodiments, wherein the pore size of the at least one filter/sieve may be in the range of 25-500 μm and the individual particle size may be in the range of 40-500 μm. In some alternate embodiments, the pore size of the at least one filter/sieve is in the range of 50-200 μm and the individual particle size is in the range of 75-200 μm.

The filtered viscous material may be collected in the second collection vessel is dispensed/injected out using a dispensing device. In some embodiments, the viscous material after filtration does not occlude the dispensing device when dispensed/injected out of the dispensing device. In some embodiments, the filtered viscous material has a substantially uniform or controlled extrusion profile when dispensed/injected out of the dispensing device.

In some embodiments, the filtered viscous material dispenses/injects out of the dispensing device without any pressure build-ups after the initial characteristic burst/break force. The individual particles of the viscous material retain their morphology i.e. they have an undamaged morphology. The filtered viscous material is capable of triggering an expected immune response in the patient upon administration.

In some embodiments, the dispensing device may be a series of injection devices, injection syringes, discharge systems, injection devices with luer-lock connectors, compressible tubes, non-compressible tubes, cylinders, or large-scale syringes. In some other embodiments, the dispensing device is a syringe, where the syringe has a needle size of 18G-34G.

In some embodiments, the at least one filter/sieve is nylon mesh, stainless steel mesh, polytetrafluoroethylene mesh or nitrocellulose mesh. The at least one filter/sieve may be placed inside, outside, adjacent to, or screwed on to the first collection vessel. The at least one filter/sieve is replaced with an inline filter, a gated impeller, a static mixer, a high shear mixer, a viscous mixer or a sieving channel. In some embodiments, the passing step sizes the individual particles of the viscous material to create a substantially uniform or controlled particulate matrix for administration.

In some embodiments the at least one filter/sieve is disposed within a luer-lock connector fitted within the first collection vessel/a luer-lock connector portion of the first collection vessel. The viscous material may be forced through the at least one filter/sieve disposed within the luer-lock connector portion under pressure/by application of pressure. The pressure may be applied using a plunger, an extruder, a manual compression, a syringe pump, a platen compression, a roller for flexible tubes, a compressor or a device capable of employing similar mechanism. In some alternate embodiments, the pressure is applied using a plunger or an extruder.

In certain embodiments, the passing step is repeated with a plurality of filters/sieves, preferably wherein the plurality of filters/sieves are of the same size or have varying sizes. The size of the at least one filter/sieve could be in the range of 1-1000 μm. In some other embodiments, passing the material through a plurality of filter/sieves reduces the viscosity or adjusts the particle size distribution of the viscous material to a desired level, preferably in the range of 50-750 μm. In some embodiments, the proposed method reduces the extrusion force required for injecting out the viscous material from the extrusion system.

In some embodiments, the viscous material could be dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

In certain embodiments, the first collection vessel is a series of vessels of varying dimensions, a storage unit or chamber, an industrial mixer, an industrial dispenser, an intermediate transfer container, a injection syringe, a large-injection syringe, a luer-lock connector device, a discharge system, or a compressible tube. In some embodiments, the second collection vessel is selected from at least one of a transfer container, a storage bottle, a injection syringe, a filler cartridge, a series of vessels of varying dimensions, a storage unit or chamber, an industrial mixer, an industrial dispenser, an intermediate transfer container, a large-injection syringe, a luer-lock connector device, a discharge system, or a compressible tube.

In some further embodiments, the second collection vessel is used for dispensing/injecting out the filtered viscous material for administration to the patient. The first collection vessel may have a volume greater than the second collection vessel

Provided is also use of any of the above method embodiments to reduce the viscosity or adjust the particle size distribution of a viscous material or a dermal filler material to a desired level, preferably in the range of 50-750 μm. Provided is also use of any of the above method embodiments to break down agglomerates/aggregates in a viscous material or a dermal filler material.

Provided further is use of any of the above method embodiments to prevent needle occlusions in an injection or dispensing device during delivery of a viscous material or dermal filler material. Provided further is also use of any of the above method embodiments to size the individual particles of a viscous or dermal filler material such that the viscous or dermal filler material has a nearly/substantially uniform individual particle size.

Provided further is also use of any of the above method embodiments to reduce the individual particle size of a viscous or dermal filler material without affecting/impacting its rheological properties. Provided further is also use of any of the above method embodiments to reduce the individual particle size of a viscous or dermal filler material without affecting/impacting its rheological properties or without affecting/impacting/changing the characteristic features or morphology of the individual particles of the viscous or dermal filler material.

Provided further is also use of any of the above method embodiments to filter a viscous or dermal material such that the viscous or dermal filler material has a uniform or controlled particulate matrix for administration. The viscous material may be selected from the group of dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

Provided further is also use of any of the above method embodiments to reduce the individual particle size of the viscous or dermal filler material thereby reducing the extrusion force required for injecting out the viscous or dermal filler material from the extrusion system.

Provided further is a device for removing agglomerations of individual particles from a viscous material that comprised an extrusion system and at least one filter/sieve placed inside, outside, adjacent to, or screwed on to the extrusion system; where passing the viscous material through the at least one filter/sieve causes removal of the agglomerations of individual particles from the viscous material.

In some embodiments, the device additionally comprises an extruder to force the viscous material through the at least one filter/sieve under pressure/by application of pressure. In some embodiments, the extruder pressure may be regulated by electronic means. The at least one filter/sieve of the device may be a nylon, stainless steel, polytetrafluoroethylene, or nitrocellulose. In some embodiments, the at least one filter/sieve may be a nylon mesh or a stainless steel mesh. In some further embodiments, the at least one filter/sieve may be replaced with an inline filter, a gated impeller, a static mixer, a high shear mixer, a viscous mixer or a sieving channel. The dimensions of the inline filter or sieving channel may be in the range of 0.5 mm to 2540 mm.

In some embodiments, the extrusion system is connected to a plurality of pre-filtering devices for pre-filtering the viscous material before passing the material through the at least one filter/sieve. The extrusion system may be connected to at least one pre-filtering device and a loading vessel/transfer syringe, where the at least one pre-filtering device is connected to the loading vessel/transfer syringe.

In some embodiments, the extrusion system is coupled to the loading vessel/transfer syringe for collecting the filtered viscous material. The at least one filter/sieve may have an aperture size smaller than the individual particle size of the viscous material. In some embodiments, the at least one filter/sieve may have an aperture size ranging from 1-1000 μm and the individual particle size is in the range of 20-1000 μm. In some alternate embodiments, the at least one filter/sieve has an aperture size ranging from 25-500 μm and the individual particle size is in the range of 40-500 μm. In some alternate embodiments, the at least one filter/sieve has an aperture size ranging from 50-200 μm and the individual particle size is in the range of 75-200 μm.

In some embodiments, the extrusion system may be an injection syringe, a discharge system, a luer-lock connector device, or a compressible tube. In some other embodiments, the extrusion system may be a syringe, where the syringe has a needle size of 18G-34G.

In some embodiments, the loading vessel/transfer syringe is selected from the group of an injection syringe, a discharge system, a luer-lock connector device, or a compressible tube. In some embodiments, the loading vessel/transfer syringe may be a syringe, where the syringe has a needle size of 18G-34G.

In some other embodiments, the extrusion system has a volume greater than the volume of the loading vessel/transfer syringe or wherein the extrusion system has a lower cross section compared to the loading vessel/transfer syringe. In some further embodiments, the extrusion system is a luer-lock connector injection device, where the at least one filter/sieve is disposed within the luer-lock connector.

Provided also is a device for removing agglomerations of individual particles from a viscous material that comprised a first collection vessel, at least one filter/sieve fitted inside, outside, adjacent to, or screwed on to the first collection vessel, wherein passing the viscous material through the at least one filter/sieve causes removal of the agglomerations of individual particles from the viscous material; and a second collection vessel coupled to the first collection vessel for collecting the filtered viscous material.

In some embodiments, the first collection vessel comprises the extrusion system as defined hereinbefore. The device of claim 169, wherein the first collection vessel is selected from the group of a series of vessels with varying dimensions, a storage unit or chamber, an industrial mixer, an industrial dispenser, an intermediate transfer container, a injection syringe, a large-injection syringe, a luer-lock connector device, a discharge system, or a compressible tube.

In some embodiment, the second collection vessel may be selected from the group of a transfer container, a storage bottle, a injection syringe, a filler cartridge, a series of vessels with varying dimensions, a storage unit or chamber, an industrial mixer, an industrial dispenser, an intermediate transfer container, a large-injection syringe, a luer-lock connector device, a discharge system, or a compressible tube. In some further embodiments, additional filter/sieve may be fitted within the second collection vessel. The second collection vessel may be a collection chamber disposed within the first collection vessel.

In certain embodiments, the device additionally comprises an extruder to force the viscous material through the at least one filter/sieve under pressure/by application of pressure, where the extruder pressure is regulated by electronic means.

In some embodiments, the at least one filter/sieve may have an aperture size ranging from 1-1000 μm and the individual particle size may be in the range of 20-1000 μm. In some alternate embodiments, the at least one filter/sieve has an aperture size ranging from 25-500 μm and the individual particle size is in the range of 40-500 μm. In some embodiments, the at least one filter/sieve has an aperture size ranging from 50-200 μm and the individual particle size is in the range of 75-200 μm.

In some embodiments, the at least one filter/sieve may be selected from a nylon mesh, a stainless steel mesh, a polytetrafluoroethylene mesh, or a nitrocellulose mesh. In some further embodiments, the at least one filter/sieve may be replaced with an inline filter, a gated impeller, a static mixer, a high shear mixer, a viscous mixer or a sieving channel.

In some embodiments, the first collection vessel may be connected to a plurality of pre-filtering devices for pre-filtering the viscous material before passing the material through the at least one filter/sieve, preferably where the first collection vessel is connected to a plurality of pre-filtering device, and the pre-filtering devices are connected to the second collection vessel. In some embodiments, the first collection vessel is connected to a first pre-filtering device and a second pre-filtering device, and the pre-filtering devices are connected to the second collection vessel.

In some embodiments, the at least one filter/sieve has an aperture size smaller than the individual particle size of the viscous material. In some embodiments, a luer-lock connector may be fitted within the first collection vessel. The at least one filter/sieve may be placed inside the luer-lock connector fitted within the first collection vessel in certain embodiments.

The experimental data is discussed in detail below.

### EXPERIMENTAL DATA

### Experiment A: Occlusion Test

In the occlusion test, the viscous material was allowed to pass through 27G and 30G needles. It was found that although some of the material passed through the needle, periodic clogging was visibly observed, which is not ideal when viscous drug formulations are being injected/administered to patients.

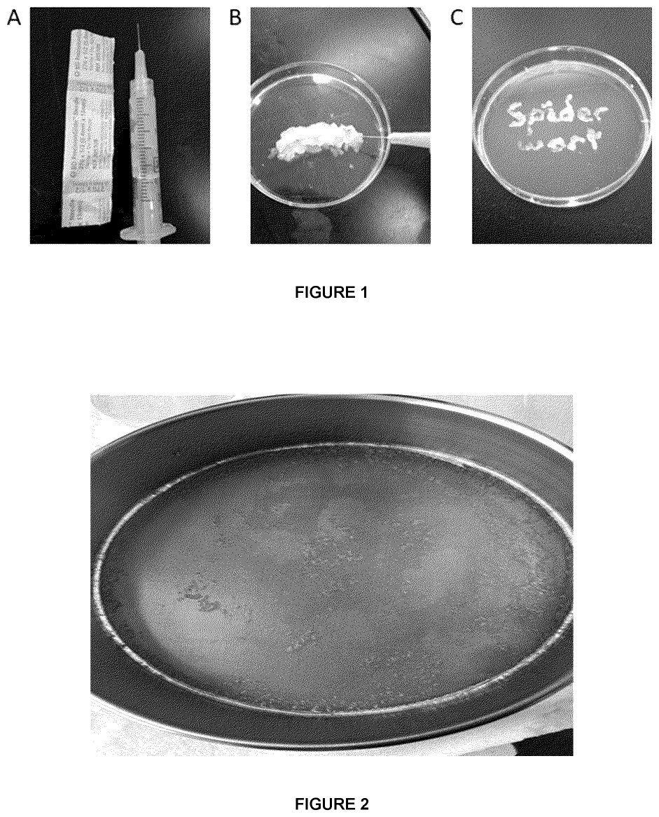

FIG. 1 shows the needle occlusion test results, wherein the viscous injectable material is passed through a 27 G needle and syringe as shown in FIG. 1 (A). FIG. 1 (B) shows the extruded mercerized viscous material and FIG. 1 (C) provides an example for 3D printing using controlled injection/extrusion techniques. Although, some viscous material passed through the needle, periodic clogging makes the technique unsuitable for drug delivery applications.

Conclusion: When the material was passed through 27 G needles, 10 ml of material occluded 10 times. When the material was passed through 30 G needles, 10.5 mL of material occluded 39 times. As is clearly apparent from the results, periodic blockage and occlusions persisted with both 27 G and 30 G needles, implying that the occlusion test failed.

### Experiment B: Sieving Test

It was originally hypothesized by the inventors that the needle occlusions were stemming from the presence of large individual particles in viscous injectable material. Therefore, the thick viscous material was diluted so that it could be filtered on a vibrating sieve. Attempts were made to pass the material through a large and a small sieve in a serial arrangement to narrow the particle size distribution. This technique is similar to differential centrifugation which is known to restrict the particle sizes to desired ranges. It is pertinent to note that occlusions didn't occur initially, however, once the material was concentrated, the occlusion issue persisted. FIG. 2 shows results of the sieving test. The Figure shows a container where individual particles/agglomerates that were too large to pass through the 425 μm sieve are being collected.

The sieving protocol is described herein. 35 mL of viscous injectable material (e.g. MerAA or CelluJuve, Ross mixed and extruded samples) was diluted into 1 L of distilled water and was stirred for 15 minutes on a magnetic stir plate. The diluted sample was sieved by placing a 425 μm sieve on top of a 45 μm sieve. 500 mL of the diluted sample was transferred onto the 425 μm sieve and vibrator was turned on for 15 seconds. Next, the 425 μm sieve was rinsed with 250 mL of distilled water, and the vibrator was turned on for 15 seconds. This step was repeated twice more. The material on the surface of the 425 μm was discarded.

Extrusion process: 1 mL of material was loaded into the syringe and a 30 G needle was affixed. The material was extruded by hand, and the number of occlusions were recorded for each 1 mL set.

The following table (Table I) shows results of the sieving test:

TABLE I
Sieved sample occlusion tally

| Syringe | Occlusions |
|---|---|
| 1 | 0 |
| 2 | 2 |
| 3 | 1 |
| 4 | 0 |
| 5 | 1 |
| 6 | 1 |
| 7 | 2 |
| 8 | 1 |
| 9 | 0 |
| 10 | 1 |
| Average | 0.9 |

Conclusion: The sieving test to create size restrictions failed to prevent needle clogging. Moreover, pressure changes were noticed during the extrusions. Therefore, sieving alone did not resolve the problems noted above.

### Experiment C: Mixing Test

The mixing apparatus was prepared with interlocked syringes connected with a luer lock adapter. Specialized mixers (planetary and static) were then used for viscous materials in an attempt to create a substantially uniform material that would not occlude the needles.

Protocol: Again, 1 mL of material was loaded into the syringe and a 30 G needle was affixed. The material was extruded by hand, and the number of occlusions were recorded for each 1 mL set. Note an occlusion event was defined as a needle blockage wherein the material ceased to flow through the needle.

The following table (Table II) shows results of the mixing test:

| Syringe | Occlusions |
|---|---|
| 1 | 2 |
| 2 | 2 |
| 3 | 1 |
| 4 | 2 |
| 5 | 0 |
| 6 | 0 |
| 7 | 1 |
| 8 | 2 |
| 9 | 1 |
| 10 | 0 |
| 11 | 2 |
| 12 | 3 |
| 13 | 1 |
| 14 | 1 |
| 15 | 0 |
| 16 | 2 |
| 17 | 1 |
| 18 | 1 |
| 19 | 2 |
| 20 | 0 |
| 21 | 1 |
| 22 | 2 |
| 23 | 3 |
| 24 | 2 |
| 25 | 2 |
| 26 | 2 |
| 27 | 1 |
| 28 | 1 |
| 29 | 1 |
| 30 | 1 |
| 31 | 2 |
| 32 | 0 |
| 33 | 0 |
| 34 | 1 |
| 35 | 1 |
| 36 | 1 |
| 37 | 1 |
| 38 | 2 |
| 39 | 2 |
| 40 | 1 |
| 41 | 3 |
| 42 | 1 |
| 43 | 3 |
| 44 | 0 |
| 45 | 1 |
| 46 | 2 |
| Average | 1.33 |

Conclusion: As is evidently clear from the table, industrial planetary and static mixers failed to prevent needle clogging.

### Experiment D: Pre-Filtration

A “brute force” method of extruding the material was attempted. The viscous material was passed through the needle until it clogged. After a clogging event, the needle was replaced with a fresh needle to remove the occlusions. The material that successfully passed through the needle was then used for occlusion testing with the same size needle used for the screening. Periodic clogging of the needles still occurred.

Conclusion: Pre-filtering via extrusion through the target needle failed to prevent needle clogging.

### Experiment E: Pre-Filtration of Mixed Sample Followed by Sieving

A combinatorial method to remove occlusions was employed. Experiments A, B and C were attempted together to assess whether an additive approach would solve the occlusion issue. The sample mixed with an industrial mixer was pre-filtered by extruding the material until the needle clogged and then replacing the needle with a fresh needle and was then diluted and sieved to remove all large clumps. The material was then collected, loaded into a syringe, and passed through the same needle size used for screening. The clogging events reduced yet significant clogging still occurred, and also the extrusions did not have a very even pressure. The results of the combinatorial method are provided in Table III below:

TABLE III
Occlusion tally for previously extruded and then sieved material.

| Syringe | Occlusions |
|---|---|
| 1 | 0 |
| 2 | 0 |
| 3 | 0 |
| 4 | 0 |
| 5 | 1 |
| 6 | 0 |
| 7 | 1 |
| 8 | 0 |
| 9 | 1 |
| 10 | 0 |
| 11 | 0 |
| 12 | 0 |
| 13 | 0 |
| 14 | 0 |
| 15 | 1 |
| 16 | 0 |
| 17 | 1 |
| 18 | 1 |
| 19 | 0 |
| 20 | 0 |
| Average | 0.30 |

Conclusion: Based on the results noted above, the inventors concluded that the combinatorial method does not provide the requisite solution as it failed to prevent needle clogging.

### Summary Statistics

To assess the success of previous experiments a statistics summary table (Table IV) was prepared. A one-way ANOVA revealed that the mixed samples (samples of Experiment B) and the mixed pre-filtered/sieved samples (samples of Experiment E) were significantly different (P=1.2×10⁻⁵). No significant differences were observed between samples of other Experiments at the 0.05 level.

TABLE IV
Summary statistics per syringe.

| Sample set | N | Mean | D | SEM |
|---|---|---|---|---|
| Planetary mixer (Experiment B) | 46 | 1.33 | 0.87 | 0.13 |
| Planetary mixer, pre-filtered, then | 20 | 0.30 | 0.47 | 0.11 |
| sieved (Experiment E) | | | | |
| Sieved (Experiment A) | 10 | 0.90 | 0.74 | 0.23 |

Conclusion: These results show that the methods presented above are not sufficient to prevent occlusions from happening. Clearly the occlusion incidence rates are too high. The risk associated with needle occlusions goes well beyond having to change needles. It may lead to an undesired amount of product being delivered under the skin, excessive force and tissue damage, apart from loss of product during extrusions.

### Experiment F: Slip Plane Modification

It was then hypothesized that creating a slip plane between the particle interfaces through addition of a lubricant could resolve the issue of needle occlusions and agglomerations. This tribology approach involved mixing the viscous injectable material with different concentrations of gelatin. However, it was found that occlusion events still occurred, and the method was not successful in removing agglomerations of individual particles from the viscous sample.

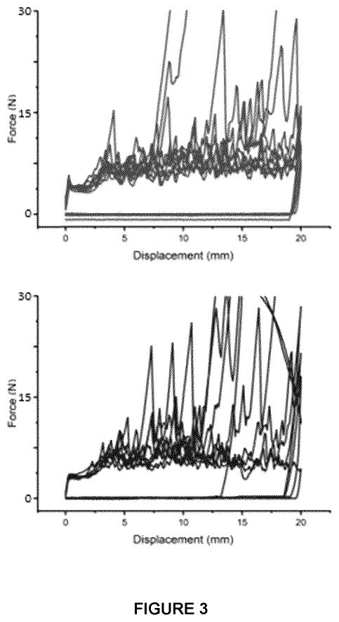

As shown in FIG. 3, from the replicate extrusion force curves and steep increases in force that overload the system (no descending portion of the curve), it is clear that addition of the lubricant (gelatin) did not prevent occlusions from occurring. It is pertinent to note that the graphs present overlayed force curves for replicate extrusions.

Conclusion: The addition of lubricant in an attempt to create a slip plane between individual particles of the viscous material failed to remove the occlusions.

### Experiment G: Pressure Drive Sieving

A solution for breaking up agglomerations without fragmentation was designed by the inventors using carefully chosen filters/sieves such as small channels or mesh screens. The method breaks up or disintegrates agglomerations by efficiently separating individual particles without affecting their desired rheological properties. The channels and mesh screens could comprise of various different materials and sizes. Although numerous combinations and prototypes are possible, the original prototype involved passing the material through 27G-30 G needles for a pre-filtering step. In some embodiments, the aperture size of the prefiltering injection device is in the range of 18G-34G. After pre-filtration, the material that passed through the needle was collected, and the material was then extruded using needles in the ranges of 18-34G for the final extrusion. The needle used in the final extrusion step had a lower gauge size compared to the needle used in the pre-filtration step. In the thick final form, the material was forced through a filter/sieve that has an aperture or pore size that was less than the average size of the material individual particles (e.g. ˜220-250 μm for the individual particles, 75-150 μm for the sieve, however the mesh sizes can range from 1-1000 μm depending on the particle size of the viscous material). In some embodiments, the individual particle size may be in the range of 20-1000 μm. It was noticed that the mercerized individual particles were able to bend and squeeze through the pores without tearing or without any damage to their morphologies or any impact on their rheological properties. Surprisingly, the small pore size effectively removed and broke up the aggregates, and thereby proved successful in removing agglomerations of individual particles from the viscous material. After small pore filtration (i.e. filtering the material through a filter with a smaller aperture size than the individual particles), no needle occlusions were found and a highly uniform extrusion profile was attained with no observable intermittent pressure build-ups and releases.

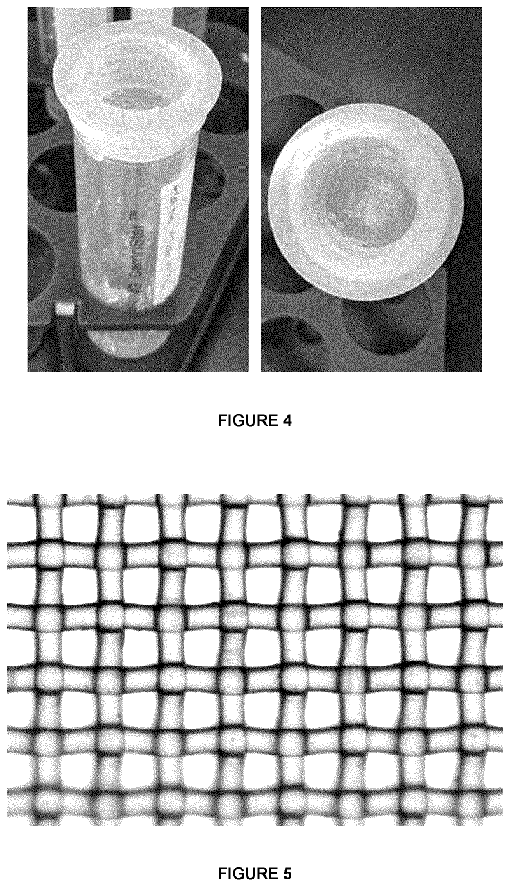

Protocol: In an exemplary embodiment, the material was pre-loaded into an injection device or extrusion system such as a syringe. The syringe was pressed firmly against a filter/sieve that was intended for use as a Falcon tube filter, as shown in FIG. 4. FIG. 5 shows a 10× magnified microscopic image of the mesh that was used in the exemplary embodiment. The material that passed through the syringe was then loaded into 1 cc syringes and was finally extruded through needles of varying sizes (in the range of 27G-33G) in 1 mL volumes, and the number of occlusion events were recorded (shown in Table V below).

TABLE V
Occlusion tally for the material that was passed through a mesh

| Syringe | Occlusions |
|---|---|
| 1 | 0 |
| 2 | 0 |
| 3 | 0 |
| 4 | 0 |
| 5 | 0 |
| 6 | 0 |
| 7 | 0 |
| 8 | 0 |
| 9 | 0 |
| 10 | 0 |
| 11 | 0 |
| 12 | 0 |
| 13 | 0 |
| 14 | 0 |
| 15 | 0 |

To analyze the efficacy of the designed process, the inventors carried out a one-way ANOVA, which revealed that the nylon mesh extruded samples were significantly different from the industrial mixer samples and the sieved samples respectively (P=4×10⁻⁸ and P=0.0126).

Although the occlusion test results were desirable, the inventors recognized that it was important to investigate the particle size and morphology, to ensure that the individual particles were not fragmented or damaged, as it would elicit an undesirable immune response in the patients upon administration.

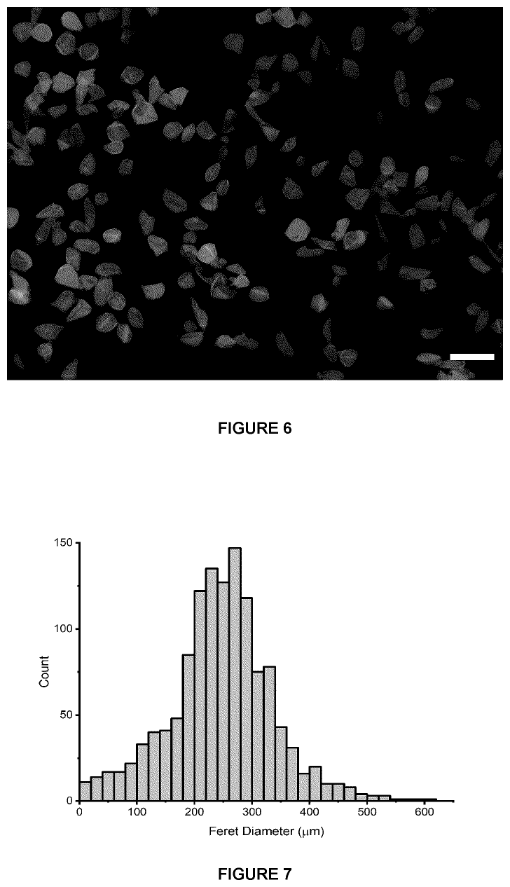

The particles were stained with 0.1% Congo Red and were imaged on an SZ16 Stereomicroscope with the BV filter as shown on the 500 μm scale as shown in FIG. 6. The particle size distribution of the particles that were passed through the mesh is shown in FIG. 7. As seen from the figure, it was found that the individual particles were not damaged and that 0.86% of the individual particles were less than 20 μm. The analysis of N=1282 individual particles resulted in a mean particle size of 246.64±2.52 μm (mean±standard error of the mean). However, this distribution was expected.

Based on the results provided in Table V, one-way ANOVA, and the staining test, the inventors concluded that passing the material through a filter/sieve (such as a mesh screen) can sufficiently break up particle agglomerations by separating individual particles and prevent clogging from happening. No occlusion events occurred when the material was extruded through an injection device or extrusion system (such as a 27G, 30 G, or 33G needle). Qualitatively, the extrusions had a very even pressure after the characteristic initial burst force. Additionally, observations made in past extrusion experiments in terms of pressure changes and pressure build-ups were not observed in this Experiment. In fact, no occlusion events were recorded and a substantially uniform extrusion was attained.

It may be argued that the proposed method for filtering and sizing individual particles of viscous injectable materials involves bringing together pressure and sieves, rather than using sieves or pressure independently. As would be appreciated by a person skilled in the art that all other “obvious” approaches failed to provide the desired extrusion profile. Additionally, the original hypothesis that high pressure sieving would simply “force” large agglomerates through the mesh, and would provide a filler material that would show increased occlusion events, was proved wrong. Moreover, it was believed that high pressure would lead to undesirable shearing of the individual particles, causing them to rip and tear. Surprisingly, the small flexible individual particles remained intact after the high local shear exposure. Moreover, the agglomeration groups were able to remain separated during extrusion rather than simply clogging the small pore of the filter/sieve.

The claimed technique not only allows sizing of the individual particles of viscous injectable materials such as dermal fillers, but also provides a viable solution for removing agglomerations while retaining the morphological characteristics and features of individual particles. As discussed earlier, a formulation with a non-uniform particle size or altered morphologies may trigger varied immune responses which is not desirable. Most importantly, the pressure sieved materials showed zero occlusion events which means there were no pressure build-ups in the needle after the initial characteristic burst/break force, which would result in expected or controlled immune responses. This also avoids the need to employ larger needles or cannulas to deliver viscous formulation, which are not only inconvenient but could also lead to unintended delivery of aggregates, which could again result in triggering uneven immune responses.

In an embodiment of the invention, the method of removing agglomerations of individual particles from a viscous material comprising the steps of a) filling the viscous material in an extrusion system comprising at least one filter/sieve; b) passing the viscous material through the filter/sieve placed inside, outside, adjacent to, inline or screwed on the extrusion system, which causes removal of the agglomerations of individual particles from the dermal filler material. In some embodiments, the viscous material is filtered by passing the filler material through an external filter/sieve or pressing the extrusion system against the filter/sieve, whereby passing the material through the sieve causes removal of the agglomerations of individual particles from the viscous material. In some embodiments, the passing step sizes the individual particles of the viscous material to create a substantially uniform or controlled particulate matrix deposition. This leads to reduction in the particle size of the viscous material without causing any severe damage to the overall morphology of the individual particles. More importantly, the individual particles retain their characteristic features, and rheological properties, and have an undamaged morphology.

In some embodiments, the filter/sieve may be a nylon mesh, a stainless steel mesh, polytetrafluoroethylene (Teflon) mesh or nitrocellulose mesh. The filter material is selected depending on the type of viscous material being filtered. For instance, stainless steel meshes can withstand higher pressures compared to nylon meshes, and therefore, can be used to filter highly viscous materials, whereas for less viscous material a nylon mesh can be used. In some embodiments, the viscous material is passed through the sieve that has a pore size or aperture size smaller than the particle size of the viscous material. The filter material and sizing can vary depending on the type of viscous material, the composition and particle size of the material, and desired viscosity of the final injectable product. The pore size of the filter/sieve is in the range of 1-1000 μm. In some embodiments, the individual particle size is in the range of 20-1000 μm. In a preferred embodiment, the pore size of the filter/sieve is in the range 25-500 μm. In such embodiments, the individual particle size could be in the range of 40-500 μm. In a further preferred embodiment, the pore size of the filter/sieve is in the range 50-200 μm. In such embodiments, the individual particle size is in the range of 75-200 μm. In an alternate embodiment, the filter/sieve is replaced with an inline filter, a gated impeller, a static mixer, a high shear mixer, a viscous mixer, a filter that is placed inside the filtering device or extrusion system, a filter that is screwed on to the extrusion system or a sieving channel. The sieving channel is a tube like structure, or a cross hatch mixing device, or a screen with a woven mesh, or a plate with perforated holes or a mesh with joined (non-woven) holes for easier cleaning.

In an embodiment of the invention, immediately after filtration, the filtered viscous material is collected in a loading vessel/transfer syringe or a second injection device. In some embodiments, the loading vessel/transfer syringe is a collection chamber, where several batches of filtered material are collected. This is more suitable for large scale applications or where bulk quantities of material are involved.

In an alternate embodiment, the loading vessel/transfer syringe or a second injection device is an injection syringe, which is suitable for direct administration to the patient. It is also possible to use the injection syringe as a collection chamber, where the filtered material is stored and subsequently transferred into smaller syringes for administration to the patient. The sizes of the loading vessel/transfer syringe may vary depending on whether it's being used as a collection device or for direct administration.

The filtered viscous material is suitable for administration to the patient as it does not occlude the loading vessel/transfer syringe when injected out of the loading vessel/transfer syringe. It is pertinent to note, that the filtered viscous material has a substantially uniform or controlled extrusion profile owing to removal or breaking-up of the agglomerations, because of which the filtered material injects out of the loading vessel/transfer syringe without any pressure build-ups. As noted earlier, the method breaks up or disintegrates agglomerations by efficiently separating individual particles without affecting their rheological properties. Even after the extrusion process, a substantial portion of the individual particles of the viscous material are intact or un-fragmented i.e. they have substantially uniform, undamaged or minimally damaged morphology after passing through the sieve. More specifically, the individual particles undergo the extrusion process without disintegrating or damaging the particles, i.e. without any impact on their characteristic features and/or morphology. This is important for triggering an expected immune response in the patient when the material is administered.

The extrusion system may be an injection syringe, a discharge system, a luer-lock connector device, or a compressible tube. If an injection syringe is preferred, the syringe can have a needle size in the range of 18-34 G. Similarly, the loading vessel/transfer syringe may be an injection syringe, a discharge system, a luer-lock connector device, or a compressible tube. If an injection syringe is preferred, the syringe of the loading vessel/transfer syringe can have a needle size in the range of 18-34G. In some embodiments, the volume of the extrusion system is similar to the volume of the loading vessel/transfer syringe. In an alternate embodiment, the volume of the extrusion system is greater than the volume of the loading vessel/transfer syringe, or wherein the extrusion system has a lower cross section compared to the loading vessel/transfer syringe. In some embodiments, there can also be a smaller syringe injecting into a larger syringe and to fill it, multiple smaller syringes or refills of a small syringe can be used. The cross sectional area is usually a consideration for how much force is required to pass it through the filter/sieve.

In some embodiments, the passing step is carried out using a luer-lock connector injection device with at least one filter/sieve disposed within the device. In such instances, the passing step additionally comprises forcing the viscous material through the sieve under pressure or by application of pressure from an external source. The pressure can be applied using a plunger, an extruder, a manual compression, a syringe pump, a platen compression, a roller, for flexible tubes or a compressor or using some similar mechanism. It is important to regulate the pressure in order to prevent unexpected mercerization of the individual particles or unnecessary damage to the particle morphology.

In an alternate embodiment, the passing step is repeated with a series of filters or sieves of varying sizes to adjust or reduce the viscosity or adjust the particle size distribution of the viscous material to a desired level, preferably in the range of 50-750 μm. In some embodiments, the method reduces the extrusion force required for injecting out the viscous material from the extrusion system. This modulation of viscosity and particle size distribution prepares the viscous material (e.g. dermal fillers) prior to its administration to patients. In such embodiments, more than one sieve/filter can be fitted within the extrusion system, such that the viscous material passes through multiple filters of varying sizes, before the material is filtered out. This can prove useful in embodiments where the viscosity of the material needs to be adjusted prior to its administration.

In an embodiment of the invention, the agglomerations present in the viscous material are removed by breaking-up the agglomerations or disintegration of the agglomerations by separating the individual particles without any impact on their characteristic features or morphology. In an alternate embodiment, the agglomerations are removed by filtering out the agglomerations that are unable to pass through the filter or sieve.

In some embodiments, the viscous material is pre-filtered through a series of pre-filtering devices prior to the passing step. The pre-filtering step can be performed using a series of injection devices, injection syringes, discharge systems, injection devices with luer-lock connectors, compressible or non-compressible tubes, cylinder, or large-scale syringes. In a preferred embodiment, the pre-filtering devices are syringes of varying aperture sizes, where the aperture sizes can range from 18-34G. The pre-filtering devices may be selected such that they are of the exact same size or have decreasing aperture sizes, depending on the material, experimental design, expected/required results, and the amount of agglomerations present in the material. In the event, decreasing sizes are preferred, the first pre-filtering device has a aperture size greater than the second pre-filtering device. Accordingly, the aperture sizes of the first and second pre-filtering device may be selected from 18-34G.

In some embodiments, the viscous material is pre-filtered through a first pre-filtering device followed by a second pre-filtering device before the passing step, i.e. before the material is forced through the sieve.

Dermal fillers are commonly employed materials for cosmetic and/or aesthetic applications such as filling wrinkles, altering appearance, or addressing soft tissue damage due to disease or injury. In certain embodiments, dermal filler products may be considered “permanent” in that they provide long-term, natural, and biocompatible solutions for such applications. Many dermal fillers in the field have been temporary. After a certain period of time, conventional dermal fillers are resorbed by the body. Others may use synthetic particles, such as PMMA, to provide longer-term solutions. In certain embodiments, the dermal fillers are cellulose-based materials processed to a target size range for dermal fillers. These may be produced from natural plant polymers, and may be permanent due to the fact that humans do not have the enzymes to break down cellulose. Therefore, the individual particles of the dermal fillers must be in the target desirable size range. The individual particles must have a desirable SA: V ratio, and desirable particle size and viscosity to efficiently promote vascularization, to contribute to biocompatibility, so that the fillers can be employed to provide for a tissue that is composed of more of the patient's own tissues rather than synthetic materials.

In an embodiment of the invention, described is a method of removing agglomerations of individual particles from a dermal filler material for administration to a patient which comprises the following steps: a) filling the dermal filler material in an extrusion system comprising at least one filter/sieve; b) passing the dermal filler material through the filter/sieve placed inside, outside, adjacent to, inline or screwed on the extrusion system, which causes removal of the agglomerations of individual particles from the dermal filler material. In some embodiments, the viscous material is filtered by passing the filler material through an external filter/sieve or pressing the extrusion system against the filter/sieve. The filtered dermal filler material is suitable for administration to the patient.

In some embodiments, the passing step sizes the individual particles of the viscous dermal filler material to create a substantially uniform or controlled particulate matrix deposition. This leads to reduction in the particle size of the viscous dermal filler material without causing any severe damage to the overall morphology of the individual particles.

In some embodiments, the filter/sieve may be a nylon mesh, a stainless steel mesh, polytetrafluoroethylene (Teflon) mesh or nitrocellulose mesh. In some embodiments, the viscous dermal filler material is passed through the sieve that has a pore size or aperture size smaller than the particle size of the viscous dermal filler material. In another embodiment, the pore size of the filter/sieve is in the range of 1-1000 μm. In such embodiments, the individual particle size is in the range of 20-1000 μm. In a preferred embodiment, the pore size of the filter/sieve is in the range 25-500 μm. In such embodiments, the individual particle size is in the range of 40-500 μm. In a further preferred embodiment, the pore size of the filter/sieve is in the range 50-200 μm. In such embodiments, the individual particle size is in the range of 75-200 μm. The sieving channel is a tube like structure, or a cross hatch mixing device, or a screen with a woven mesh, or a plate with perforated holes or a mesh with joined (non-woven) holes for easier cleaning.

In an embodiment of the invention, immediately after filtration, the filtered viscous dermal filler material is collected in a loading vessel/transfer syringe. In some embodiments, the loading vessel/transfer syringe is a collection chamber, where several batches of filtered filler material are collected. This is more suitable for large scale applications or where bulk quantities of filler material are involved.

In an alternate embodiment, the loading vessel/transfer syringe is an injection or collection syringe, which is suitable for direct administration to the patient. It is also possible to use the injection syringe as a collection chamber, where the filtered filler material is stored and subsequently transferred into smaller syringes for administration to the patient. The sizes of the loading vessel/transfer syringe may vary depending on whether it's being used as a collection device or for direct administration.

The filtered viscous dermal filler material is suitable for administration to the patient as it does not occlude the loading vessel/transfer syringe when injected out of the loading vessel/transfer syringe. It is pertinent to note, that the filtered viscous dermal filler material has a substantially uniform or controlled extrusion profile owing to separation of individual particles leading to removal or breaking-up of the agglomerations, because of which the filtered filler material injects out of the loading vessel/transfer syringe without any pressure build-ups. The agglomerations are broken down by separation of individual particles without affecting their morphological properties. Even after the extrusion process, a substantial portion of the individual particles of the viscous dermal filler material are intact or un-fragmented i.e. they have uniform, undamaged or minimally damaged morphology after passing through the sieve. In some embodiments, at least 98%, at least 95%, at least 92%, at least 89%, at least 86%, at least 83%, or at least 80% of the individual particles of the viscous material pass through the filter/sieve without affecting their characteristic features or morphology i.e. the method does not change the characteristic features or morphology of at least 98%, at least 95%, at least 92%, at least 89%, at least 86%, at least 83%, or at least 80% of the individual particles of the viscous material. This is important for triggering an expected immune response in the patient when the filler material is administered.

The extrusion system may be an injection syringe, a discharge system, a luer-lock connector device, or a compressible tube. If an injection syringe is preferred, the syringe can have a needle size in the range of 18-34G. Similarly, the loading vessel/transfer syringe may be an injection syringe, a discharge system, a luer-lock connector device, or a compressible tube. If an injection syringe is preferred, the syringe of the loading vessel/transfer syringe can have a needle size in the range of 18-34G. In some embodiments, the volume of the extrusion system is similar to the volume of the loading vessel/transfer syringe. In an alternate embodiment, the volume of the extrusion system is greater than the volume of the loading vessel/transfer syringe, or wherein the extrusion system has a lower cross section compared to the loading vessel/transfer syringe. In some embodiments, there can also be a smaller syringe injecting into a larger syringe and to fill it, multiple smaller syringes or refills of a small syringe can be used. The cross sectional area is usually a consideration for how much force is required to pass it through the filter/sieve.

In some embodiments, the passing step is carried out using a luer-lock connector injection device with at least one filter/sieve disposed within the device. In such instances, the passing step additionally comprises forcing the viscous dermal filler material through the sieve under pressure or by application of pressure from an external source. The pressure can be applied using a plunger, an extruder, a manual compression, a syringe pump, a platen compression, a roller, for flexible tubes or a compressor or using some similar mechanism. It is important to regulate the pressure in order to prevent unexpected fragmentation of the individual particles or unnecessary damage to the particle morphology.

In an alternate embodiment, the passing step is repeated with a series of filters or sieves of varying sizes to adjust or reduce the viscosity or the particle size distribution of the viscous dermal filler material to a desired level, preferably in the range of 50-750 μm. In such embodiments, more than one sieve/filter can be fitted within the extrusion system, such that the viscous dermal filler material passes through multiple filters of varying sizes, before the filler material is filtered out. This can prove useful in embodiments where the viscosity of the filler material needs to be adjusted prior to its administration. This modulation of viscosity and particle size distribution prepares the viscous material (e.g. dermal fillers) prior to its administration to patients.

In an embodiment of the invention, the agglomerations present in the viscous dermal filler material are removed by separation of individual particles that breaks-up the agglomerations or disintegration of the agglomerations. In an alternate embodiment, the agglomerations are removed by filtering out the agglomerations that are unable to pass through the filter or sieve.

In some embodiments, the viscous dermal filler material is pre-filtered through a series of pre-filtering devices, wherein the pre-filtration step occurs prior to the passing step. The pre-filtering step can be performed using a series of injection devices, injection syringes, discharge systems, injection devices with luer-lock connectors, compressible or non-compressible tubes, cylinder, or large-scale syringes. In a preferred embodiment, the pre-filtering devices are syringes of varying needle sizes, where the pre-filtering devices may be selected such that they are of the exact same size or have decreasing needle sizes, depending on the filler material, experimental design, expected/required results, and the amount of agglomerations present in the filler material. In the event, decreasing sizes are preferred, the first pre-filtering device has an aperture size greater than the second pre-filtering device. Accordingly, the aperture sizes of the first or second pre-filtering device may be selected from 18-34G.

In some embodiments, the viscous dermal filler material is pre-filtered through a first pre-filtering device followed by a second pre-filtering device before the passing step, i.e. before the filler material is forced through the sieve.

In an embodiment of the invention, the method of removing agglomerations of individual particles from the viscous material may comprise the following steps: a) filling the viscous material in a first collection vessel comprising at least one filter/sieve; b) passing the viscous material through the filter/sieve causing removal of the agglomerations of individual particles from the viscous material; and c) collecting the viscous material in a second collection vessel. In some embodiments, the first collection vessel may comprise the extrusion system as defined hereinbefore, and the extrusion system comprises the filter/sieve. The filtered viscous material collected in the second collection vessel is suitable for administration to the patient. The collection vessels may have mixing capacity in the range of 0.02 gallons to 750 gallons.

Without wishing to be bound by theory, the following charts provide some examples of mixers and vessels currently offered by Charles Ross & Company that could be employed for large scale filtering operations. A person skilled in the art would readily understand that there could be other vessels that could be used for the filtering operations.

Exemplary Double Planetary Mixers offered by Charles Ross & Son Company

| | | | | | Raised | Lowered | | | Mix Vessel | Mix Vessel | Mix | |
|---|---|---|---|---|---|---|---|---|---|---|---|---|
| | Mixing | | | | Overall | Overall | Overall | Overall | Overall | Inside | Vessel | |
| | Capacity | | Orbital | Stirrer | Height | Height | Width | Length | Height | Diameter | Depth | Weight |
| Model | gallons | HP | RPM | RPM | inches | inches | inches | inches | inches | inches | inches | lbs |
| DPM-½Pt | 0.02-0.06 | ½ | 9-88 | 9-88 | 25.5 | N/A | 12 | 16 | 3 | 3.5 | 2.5 | 165 |
| DPM-1Pt | 0.06-0.13 | ½ | 7-72 | 8-78 | 25.5 | N/A | 12 | 20 | 4.25 | 5.75 | 3.25 | 190 |
| DPM-1Qt | 0.06-0.25 | ½ | 9-88 | 10-100 | 29.00 | N/A | 13.5 | 21.38 | 5 | 5.88 | 4 | 200 |
| DPM-1 | 0.2-0.75 | 1.5 | 5-100 | 9-180 | 31.25 | 24.25 | 23 | 33.25 | 6.5 | 8.25 | 5 | 390 |
| DPM-2 | 0.2-1.5 | 1.5 | 5-97 | 6-122 | 32.25 | 24.88 | 23 | 35.25 | 8 | 9.63 | 6.5 | 450 |
| DPM-4 | 0.5-4 | 2 | 4-69 | 4-80 | 41.25 | 32.25 | 23 | 43.25 | 9.63 | 14 | 8 | 520 |
| DPM-5 | 0.5-5 | 2 | 4-69 | 4-80 | 43.25 | 34.25 | 25 | 45.25 | 11.63 | 16 | 10 | 580 |
| DPM-10 | 2-12 | 3-5 | 11-45 | 12-48 | 80 | 65 | 32.5 | 65 | 16.3 | 18 | 13.5 | 2,500 |
| DPM-25 | 5-25 | 5-20 | 8-33 | 9-36 | 107 | 84 | 35.5 | 74 | 25.4 | 25 | 16 | 4,200 |
| DPM-40 | 5-40 | 5-20 | 8-33 | 9-36 | 107 | 84 | 35.5 | 74 | 25.4 | 25 | 22 | 4,200 |
| DPM-75 | 10-75 | 10-30 | 8-30 | 9-34 | 149 | 115 | 44 | 96 | 31.8 | 34.5 | 22.5 | 9,000 |
| DPM-100 | 10-100 | 10-30 | 8-30 | 9-34 | 149 | 115 | 44 | 96 | 31.8 | 34.5 | 28.5 | 9,000 |
| DPM-150 | 20-150 | 20-40 | 6-24 | 7-26 | 166 | 132 | 50 | 115 | 37 | 44 | 28 | 13,500 |
| DPM-200 | 20-200 | 20-75 | 6-24 | 7-26 | 166 | 132 | 50 | 115 | 37 | 44 | 34 | 13,500 |
| DPM-300 | 75-300 | 50-100 | 6-22 | 7-28 | 196 | 156 | 114 | 78 | 41 | 55.5 | 38 | 25,000 |
| DPM-400 | 100-400 | 50-100 | 6-22 | 7-28 | 255 | 208 | 114 | 78 | 48 | 55.5 | 45 | 28,000 |
| DPM-500 | 150-500 | 150 | 4-16 | 5-20 | 259 | 211 | 122 | 108 | 48 | 66 | 42 | 38,000 |
| DPM-750 | 150-750 | 150 | 4-16 | 5-20 | 294 | 228 | 122 | 108 | 66 | 66 | 60 | 41,000 |

In some embodiments, the first collection vessel is a series of vessels with varying dimensions, a storage unit or chamber, an industrial mixer, an industrial dispenser, an intermediate transfer container, an injection syringe, a large-injection syringe, a luer-lock connector device, a discharge system, or a compressible tube. The second collection device may be selected from at least one of a transfer container, a storage bottle, a injection syringe, a filler cartridge, a series of vessels with varying dimensions, a storage unit or chamber, an industrial mixer, an industrial dispenser, an intermediate transfer container, a large-injection syringe, a luer-lock connector device, a discharge system, or a compressible tube.

The method of removing agglomerations using collection vessels does not change the characteristic features or morphology of at least 98%, at least 95%, at least 92%, at least 89%, at least 86%, at least 83%, or at least 80% of the individual particles of the viscous material i.e. at least 98%, at least 95%, at least 92%, at least 89%, at least 86%, at least 83%, or at least 80% of the individual particles of the viscous material pass through the filter/sieve without affecting their characteristic features or morphology.

In some embodiments, the viscous material is forced the viscous material through the filter/sieve under pressure/by application of pressure using a plunger, an extruder, a manual compression, a syringe pump, a platen compression, a roller, or a compressor, or using a similar mechanism.

Without wishing to be bound by theory, it is believed that the agglomerations are removed from the viscous material by breaking-up/disintegrating the agglomerations or by filtering out/sieving out the agglomerations that are unable to pass through the filter/sieve. The method allows separation of individual particles without affecting their rheological properties which causes the agglomerations to disintegrate. The passing step sizes the individual particles of the viscous material to create a substantially uniform or controlled particulate matrix which is suitable for administration to a patient in need thereof.

In some embodiments, the viscous material is pre-filtered through a plurality of pre-filtering devices, wherein the pre-filtration step occurs prior to the passing step. In an alternate embodiment the viscous material is pre-filtered through a first pre-filtering device followed by a second pre-filtering device, where the plurality of pre-filtering devices are syringes of varying aperture sizes. pre-filtering step can be performed using a series of injection devices, injection syringes, discharge systems, injection devices with luer-lock connectors, compressible or non-compressible tubes, cylinder, or large-scale syringes. In some embodiments, the first pre-filtering device has a aperture size greater than the second pre-filtering device. The aperture size of the first and second pre-filtering device can be selected from 18-34G.

In some embodiments, the viscous material is passed through at least one filter/sieve having a pore size/an aperture size smaller than the particle size of the viscous material, where the pore size of the filter/sieve is in the range of 1-1000 μm. In such embodiments, the individual particle size could be in the range of 20-1000 μm. In some embodiments, the pore size of the filter/sieve is in the range of 25-500 μm or in the range of 50-200 μm. In such embodiments, the individual particle size could be in the range of 40-500 μm or 75-200 μm.

The filtered viscous material may be collected in the second collection vessel is dispensed/injected out using a dispensing device, where the filtered viscous material does not occlude the dispensing device when dispensed/injected out of the dispensing device. The filtered viscous material has a substantially uniform or controlled extrusion profile when dispensed/injected out of the dispensing device, where the filtered viscous material dispenses/injects out of the dispensing device without any pressure build-ups after the initial characteristic burst/break force.

In some embodiments, the dispensing device is an industrial filtration unit, large injection devices, screw driven extruder, hydraulic platen pump or an electric discharge system In a preferred embodiment, the dispensing device is a syringe, wherein the syringe has a needle size of 18-34G In some embodiments, the filter/sieve is a nylon mesh, stainless steel mesh, polytetrafluoroethylene mesh or nitrocellulose mesh. In an alternate embodiment, the filter/sieve is replaced with an inline filter, a gated impeller, a static mixer, a high shear mixer, a viscous mixer, a filter that is placed inside the filtering device, a filter that is screwed on to the filtering device or a sieving channel.

In some embodiments, the filter/sieve is disposed within a luer-lock connector fitted within the first collection vessel/a luer-lock connector portion of the first collection vessel, where the viscous material is forced through the filter/sieve disposed within the luer-lock connector portion under pressure/by application of pressure. In such embodiments, where the pressure is applied using a plunger, an extruder, a manual compression, a syringe pump, a platen compression, a roller, for flexible tubes or a compressor or using some similar mechanism.

In some embodiments, the passing step may be repeated with a plurality of filters/sieves to adjust or reduce the viscosity or the particle size distribution of the viscous material to a desired level, preferably in the range of 50-750 μm, where the plurality of filters/sieves are of the same size or have varying sizes in the range of 1-1000 μm. This modulation of viscosity and particle size distribution prepares the viscous material (e.g. dermal fillers) prior to its administration to patients. The method reduces the extrusion force required for injecting out the viscous material from the extrusion system.

In some embodiments, the second collection vessel is used for dispensing/injecting out the filtered viscous material for administration to the patient, where the first collection vessel has a volume greater than the second collection vessel.

In some embodiments, the methods described hereinbefore can be used to adjust the viscosity of viscous materials or dermal filler materials to a desired level. In some embodiments, the methods described hereinbefore can be employed for breaking down agglomerates/aggregates in a viscous material or a dermal filler material. The methods described can also prove useful when preventing needle occlusions in an injection device is of material importance, especially during delivery of a viscous material or any dermal filler material. Furthermore, the described methods can help in sizing the individual particles of a viscous material such that the viscous material or dermal filler material has a nearly uniform particle size. Additionally, the methods may be applied to reduce the particle size of viscous or dermal filler materials without affecting/impacting their rheological properties, such that the viscous or dermal filler materials have a substantially uniform or controlled particulate matrix deposition. In some embodiments, the method reduces the extrusion force required for injecting out the viscous material from the extrusion system.

The methods described can be used for a broad range of viscous materials. Without limiting the scope of its application, the methods can be used to treat viscous materials such as dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers. The methods described can also be used to remove agglomerations from other material such as Gum and Hydrocolloids, Cassia gum powder, Guar Gum Powder, Fast Hydration Guar Gum Powder, Cassia Tora Powder, Tamarind Kernel Powder, Sesbania Gum Powder, Fenugreek Gum Powder, Psyllium Husk Powder, Kappa Carrageenan Gum Powder, Locust Bean Gum Powder, Date palm mucilage, Guar Meal, “Erva Baleeira” Mucilage, Guar gum, Gum kondagogu, Konjac glucomannan, Taro, Gellan gum, Curdlan gum, Starches, Wheat Starch, Maize Starch, Barley Starch, Modified Starches, Arrowroot Starch, Cornstarch, Tapioca Powder, Potato Starch, Stabilizers, Acacia (Gum Arabic), Agar-agar, Ammonium alginate, Calcium alginate, Carob bean gum (Locust bean gum), Chondrus extract (Carrageenan), Ghatti gum, Guar gum, Pectin, Potassium alginate, Sodium alginate, Sterculia gum (Karaya gum), Tragacanth (gum tragacanth), Thickener, Sodium alginate, Potassium alginate, Ammonium alginate, Calcium alginate, Carrageenan, Locust bean gum, Guar gum, Karaya gum, Gellan gum, Mannitol, Konjac, Pectins, Cellulose, Methylcellulose, Hydroxypropyl cellulose, Hydroxypropyl methylcellulose, Isomalt, Maltitol, Xylitol, Oxydised starch, Monostarch phosphate, Distarch Phosphate, Gelling agent, Alginic acid, Sodium alginate, Potassium alginate, Agar, Carrageenan, Locust bean gum, Gellan gum, Konjac, Pectins, Cellulose, Methylcellulose, Hydroxypropyl cellulose, and Hydroxypropyl methylcellulose.

In some embodiments, the methods can be used to reduce the particle size of the materials thereby reducing the extrusion force required for injecting out the viscous material from the extrusion system. In some embodiments, the methods can help in reducing viscosity of viscous or dermal filler materials in the range of 1-99%. In some embodiments, the methods can be used to filter and extrude viscous materials using controlled or uniform extrusion force.

In some embodiments, described is a device for removing agglomerations of individual particles from a viscous material which comprises the following elements: a) a extrusion system; and b) at least one filter/sieve placed inside, outside, adjacent to, inline or screwed on the extrusion system, which causes removal of the agglomerations of individual particles from the dermal filler material. In some embodiments, the viscous material is filtered by passing the filler material through an external filter/sieve or pressing the extrusion system against the filter/sieve, which allows removal of agglomerations for viscous/dermal filler materials when the material is passed through the filter/sieve.

In some embodiment, the device may additionally comprise an extruder to force the viscous/dermal filler material through the filter/sieve under pressure. The extruder pressure can be regulated by electronic or mechanical means.

The filter/sieve can be selected from nylon mesh, a stainless steel mesh, polytetrafluoroethylene (Teflon) mesh or nitrocellulose mesh. In some embodiments, the filter/sieve is a nylon mesh. The nylon mesh used in the process could be replaced with a more robust solution for large-scale operations. For example, an in-line 316 stainless steel mesh (in several different size options) can be used instead of nylon mesh.

The filter/sieve may have a pore size in the range of 1-1000 μm. In such embodiments, the individual particle size could be in the range of 20-1000 μm. In a preferred embodiment, the pore size of the filter/sieve is in the range 25-500 μm. In such embodiments, the individual particle size could be in the range of 40-500 μm In a further preferred embodiment, the pore size of the filter/sieve is in the range 50-200 μm. In such embodiments, the individual particle size could be in the range of 75-200 μm In an alternate embodiment, the filter/sieve is replaced with an inline filter, a gated impeller, a static mixer, a high shear mixer, a viscous mixer, a filter that is placed inside the filtering device, a filter that is screwed on to the filtering device or a sieving channel. The dimensions of the inline filter could be in the range of 0.5 mm-2540 mm. The sieving channel is a tube like structure, or a cross hatch mixing device, or a screen with a woven mesh, or a plate with perforated holes or a mesh with joined (non-woven) holes for easier cleaning.

In an alternate embodiment, the design of the device could be altered such that the extrusion system is connected to a series of pre-filtering devices such that the viscous or dermal filler material is for pre-filtered at least before passing through the filter/sieve. In such embodiments, the extrusion system is connected to at least one pre-filtering device and the pre-filtering device(s) is/are linked to the loading vessel/transfer syringe. The loading vessel/transfer syringe could simply be a collection chamber to collect and store the filtered material, especially in cases where multiple batches of material are being filtered/pre-filtered. In some additional embodiments, the loading vessel/transfer syringe could be used for administration of the material to the patient.

The extrusion system may be an injection syringe, a discharge system, a luer-lock connector device, or a compressible tube. The loading vessel/transfer syringe may be an injection syringe, a discharge system, a luer-lock connector device, or a compressible tube. The at least one pre-filtering devices may be a series of injection devices, syringes, discharge systems, injection devices with luer-lock connectors, compressible or non-compressible tubes, cylinder, or large-scale syringes. The injection devices may have a needle size of ranging from 18-34G. In some embodiments, the extrusion system may have a volume greater than the volume of the loading vessel/transfer syringe, or wherein the extrusion system has a lower cross section compared to the loading vessel/transfer syringe. For example, the extrusion system and loading vessel/transfer syringe could be 1 ml syringes, or in an alternate embodiment, the extrusion system could be a 5 ml syringe and the transfer syringe could be a 1 ml syringe. In such cases, the aperture size of the extrusion system could be greater than the aperture size of the loading vessel/transfer syringe. In some embodiments, there can also be a smaller syringe injecting into a larger syringe and to fill it, multiple smaller syringes or refills of a small syringe can be used. The cross sectional area is usually a consideration for how much force is required to pass it through the filter/sieve.

In some embodiments, the filter/sieve used for the final passing/filtration step has an aperture size smaller than the particle size of the viscous material. The smaller size helps in mercerization of the particles to achieve a substantially uniform or controlled particulate distribution. The aperture size of the filter/sieve may range from 1-1000 μm.

In some embodiments, a luer-lock connector may be fitted within the extrusion system. The luer lock connector can house the filter/sieve i.e. the filter/sieve can be placed in the luer-lock connector, instead of placing it at the bottom of the first-injection device.

In an alternate embodiment, the device may have a slightly different configuration, wherein the device for removing agglomerations may comprise a) a first collection device; b) at least one filter/sieve fitted inside the first collection device through which the viscous/dermal filler material is passed causing removal of the agglomerations of individual particles from the material; and c) a second collection device coupled to the first collection device for collecting the filtered material.

In such embodiments, the first collection device may be a series of vessels with varying dimensions, a storage unit or chamber, an industrial mixer, an industrial dispenser, an intermediate transfer container, a luer-lock connector device, a injection syringe, a large-injection syringe, a discharge system, or a compressible tube. In an alternate embodiment, the second collection device may be a transfer container, a storage bottle, a injection syringe, a filler cartridge, a series of vessels with varying dimensions, a storage unit or chamber, an industrial mixer, an industrial dispenser, an intermediate transfer container, a large-injection syringe, a luer-lock connector device, a discharge system, or a compressible tube.

In some embodiments, an additional filter/sieve is fitted within the second collection device. In such embodiments, the aperture size of the first and additional filter/sieve(s) may be in the range of 1 to 1000 microns. In such embodiments, the individual particle size could be in the range of 20-1000 μm. In a preferred embodiment, the pore size of the filter/sieve is in the range 25-500 μm. In such embodiments, the individual particle size could be in the range of 40-500 μm. In a further preferred embodiment, the pore size of the filter/sieve is in the range 50-200 μm. In such embodiments, the individual particle size could be in the range of 75-200 μm. The filter/sieve(s) may be a nylon, stainless steel, Teflon, or nitrocellulose. Is some preferable embodiments, the filter/sieve may be a nylon mesh or a stainless steel mesh. In an alternate embodiment, the filter/sieve is replaced with an inline filter, a gated impeller, a static mixer, a high shear mixer, a viscous mixer, a filter that is placed inside the filtering device, a filter that is screwed on to the filtering device or a sieving channel. The dimensions of the inline filter could be in the range of 0.5 mm-2540 mm. The sieving channel is a tube like structure, or a cross hatch mixing device, or a screen with a woven mesh, or a plate with perforated holes or a mesh with joined (non-woven) holes for easier cleaning.

In some configurations, the second collection device is a collection chamber disposed within the first collection device. In such embodiments, after passing through the filter/sieve fitted within the first collection device, the viscous material is stored in a chamber (i.e. the second collection device which is fitted within the first collection device.

In some embodiment, the device may additionally comprise an extruder to force the viscous/dermal filler material through the filter/sieve under pressure. The extruder pressure can be regulated by electronic or mechanical means. Alternatively, the pressure can be applied using a plunger, an extruder, a manual compression, a syringe pump, a platen compression, a roller, for flexible tubes or a compressor or using some similar mechanism. It is important to regulate the pressure in order to prevent unexpected mercerization of the particles or unnecessary damage to the particle morphology. The extruder pressure can be regulated by electronic or mechanical means.

In an alternate embodiment, the design of the device could be altered such that the first collection device is connected to a series of pre-filtering collection devices such that the viscous or dermal filler material is for pre-filtered at least before passing through the filter/sieve. In such embodiments, the first collection device is connected to at least one pre-filtering collection device and the pre-filtering device(s) is/are linked to the second collection device. The second collection device could simply be a collection chamber to collect and store the filtered material, especially in cases where multiple batches of material are being filtered/pre-filtered. The material collected can be transferred to dispensing/administration/injection devices or syringes for final administration of the filtered material to the patient.

The first collection device may be a series of vessels with varying dimensions, a storage unit or chamber, a luer-lock connector device, an industrial mixer, an industrial dispenser, an intermediate transfer container, a injection syringe, a large-injection syringe, a discharge system, or a compressible tube. The second collection device may be a transfer container, a storage bottle, a injection syringe, a filler cartridge, a series of vessels with varying dimensions, a storage unit or chamber, an industrial mixer, an industrial dispenser, an intermediate transfer container, a large-injection syringe, a luer-lock connector device, a discharge system, or a compressible tube. The at least one pre-filtering devices may be a series of injection devices, syringes, discharge systems, injection devices with luer-lock connectors, compressible or non-compressible tubes, cylinder, or large-scale syringes. The dispensing/administration/injection device may be a series of injection devices, injection syringes, luer-lock syringe, large-scale syringes, and may have a needle size of ranging from 18-34G.

Similar to other device configuration, in some embodiments, the filter/sieve used for the final passing/filtration step has an aperture size smaller than the particle size of the viscous material. The smaller size helps in mercerization of the particles to achieve a substantially uniform or controlled particulate distribution. The aperture size of the filter/sieve may range from 1 to 1000 μm.

In some embodiments, a luer-lock connector may be fitted within the first collection device. The luer lock connector can house the filter/sieve i.e. the filter/sieve can be placed inside the luer-lock connector, instead of placing it at the bottom of the first-collection device.

### Potential Solution and Configurations

The invention will now be described in terms of exemplary potential configurations that may be employed for carrying out the pressure sieving process of removing agglomerations of individual particles from a given viscous material. There are multiple potential applications of the claimed technique such as filtering agglomerates from drug formulations, dermal filler applications, soft tissue implants, lubricants and vehicles for small peptide or cell delivery.

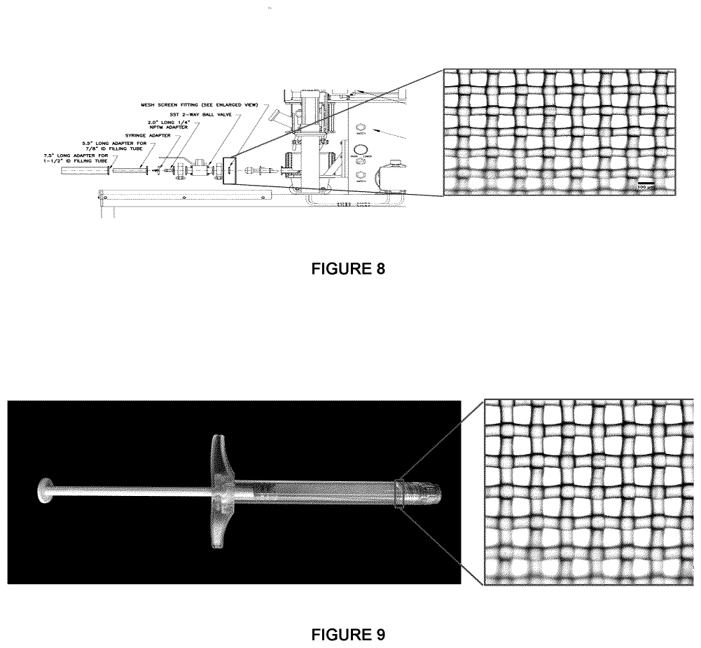

FIG. 8 shows an in-line pressure filtration process that can be employed for large scale implementation of the pressure filtration/sieving process. The in-line pressure filtration system comprises a discharge system which is used to force the viscous material through a mesh screen. In an implementation of the in-line process, the mesh screen can be fitted between the discharge system (injection device) and a collection vessel such a loading vessel. The schematic of the in-line mesh screen shown in the Figure is provided by Charles Ross & Son Company (left portion of the Figure). The filtered viscous material is collected in the collection vessel prior to being transferred in smaller transfer syringes.

As shown in the figure, the screen can be a mesh sieve, which is either pre-fitted in the discharge system or can be fitted prior to the filtration process. The syringe can be placed between a syringe adapter followed by a filling tube extension and the discharge system. As noted earlier, this configuration is more suited for highly viscous materials or where several batches of materials are being treated at the same time.

In general, the discharge system comprises a large vessel, platen or extruder, a valve and an extrusion port, and when the valve is opened, the platen pushes the material out through the extrusion port.

FIG. 9 shows an in-syringe process configuration for treating viscous materials and removal of agglomerations. In the in-syringe process, an injection device pre-fitted with a filter/sieve is used. A smaller version of the filter, as used in the in-pressure filtration process, is placed towards the bottom of the injection device on the inside such that the viscous material passes through the filter/sieve before exiting the injection device. The filtered material can be collected in a loading vessel/transfer syringe or a separate collection vessel. In this configuration, the filtration force is supplied by the extruder. The extruder force forces the material through the filter/sieve such that the material passes through the filter and is filtered before exiting the injection device. In some embodiments, the filtered material can be administered to the patients by collecting them in small transfer syringes. In such instances, there's no collection device to collect the filtered viscous material as the material is administered to the patients directly using transfer syringes. In alternate embodiments, the filtered material can be stored in a separate collection vessel. This configuration i.e. the in-syringe process is more suited for small scale applications.

In some embodiments, the injection device is a syringe with a needle size of 18G-34G. The filter/sieve placed at the base of the syringe can be selected from a nylon mesh screen, a stainless steel mesh screen, polytetrafluoroethylene (Teflon) mesh screen or nitrocellulose mesh screen. The loading vessel/transfer syringe can be an injection syringe, a discharge system, a luer-lock connector device, or a compressible tube. The types of viscous material that can be filtered using this process is dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers. The methods described can also be used to remove agglomerations from other material such as Gum and Hydrocolloids, Cassia gum powder, Guar Gum Powder, Fast Hydration Guar Gum Powder, Cassia Tora Powder, Tamarind Kernel Powder, Sesbania Gum Powder, Fenugreek Gum Powder, Psyllium Husk Powder, Kappa Carrageenan Gum Powder, Locust Bean Gum Powder, Date palm mucilage, Guar Meal, “Erva Baleeira” Mucilage, Guar gum, Gum kondagogu, Konjac glucomannan, Taro, Gellan gum, Curdlan gum, Starches, Wheat Starch, Maize Starch, Barley Starch, Modified Starches, Arrowroot Starch, Cornstarch, Tapioca Powder, Potato Starch, Stabilizers, Acacia (Gum Arabic), Agar-agar, Ammonium alginate, Calcium alginate, Carob bean gum (Locust bean gum), Chondrus extract (Carrageenan), Ghatti gum, Guar gum, Pectin, Potassium alginate, Sodium alginate, Sterculia gum (Karaya gum), Tragacanth (gum tragacanth), Thickener, Sodium alginate, Potassium alginate, Ammonium alginate, Calcium alginate, Carrageenan, Locust bean gum, Guar gum, Karaya gum, Gellan gum, Mannitol, Konjac, Pectins, Cellulose, Methylcellulose, Hydroxypropyl cellulose, Hydroxypropyl methylcellulose, Isomalt, Maltitol, Xylitol, Oxydised starch, Monostarch phosphate, Distarch Phosphate, Gelling agent, Alginic acid, Sodium alginate, Potassium alginate, Agar, Carrageenan, Locust bean gum, Gellan gum, Konjac, Pectins, Cellulose, Methylcellulose, Hydroxypropyl cellulose, and Hydroxypropyl methylcellulose.

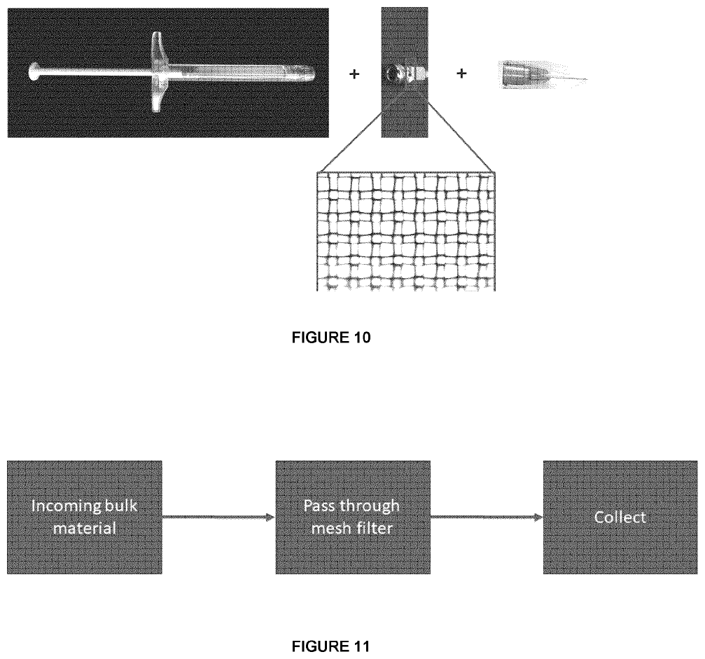

FIG. 10 shows an in-line syringe process configuration where a small version of the filter/sieve is placed between the needle and the syringe in a custom luer lock connector device. A luer-lock connector syringe is capable of providing leak proof transfer of material between the syringe and the needle, and most importantly, it provides protection against accidental removal or the needle or leakage of the material content by accident. In this configuration, the filtration force can also be supplied by an extruder, and similar to the in-line syringe process, the material is filtered immediately before reaching the needle. In this configuration, the filter is not placed in the syringe. The intermediate luer lock connection provides simple and rapid attachment of the filter/sieve.

The treated/filtered material poses a significant filling challenge due to its high viscosity. In order to avoid compromising the end cap seal of the injection device/syringe as well as preventing excessive silicone shedding from multiple plunger passes, the material can be back-loaded with large transfer needles, specifically 8-10 G needles. The stopper is then placed with commercially available vent-tube stoppering methods

The overarching concept of the invention is to use pressure driven filtration to break up agglomerations and filter out large individual particles. This concept can be applied at multiple levels or scales. In its simplest form, the process involves passing the bulk material through small openings, such as a mesh screen or perforated sheet, and then collecting the filtered material in smaller syringes or collection vessels. This concept of the filtration process is shown in the form of a block diagram in FIG. 11.

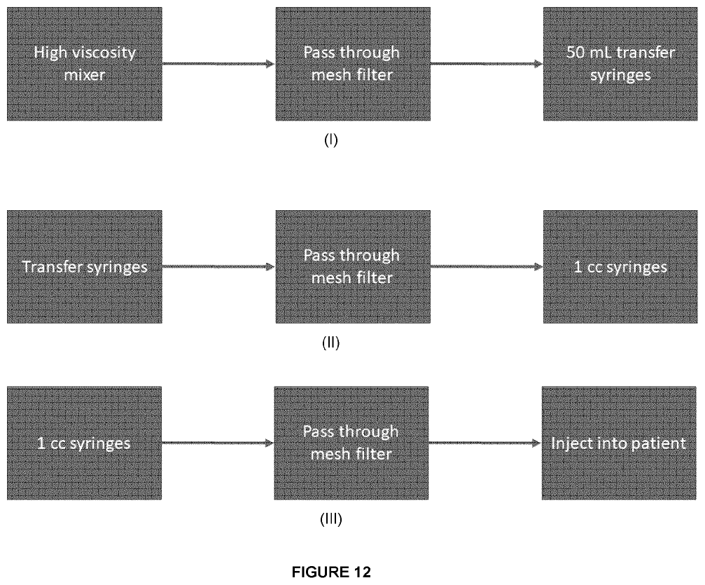

This inventive technique can be applied multiple times and/or in multiple scales. FIG. 12 provides a block diagram showing how the process can be applied in a large, medium or small scale.

FIG. 12 (I) shows an exemplary large scale operation. In a large scale operation, the incoming material (or the material to be treated) is in bulk quantities. A large batch of material can be treated in an industrial high viscosity mixer/dispenser, wherein the filter/sieve can be an inline filter, a gated impeller, a static mixer, a high shear mixer, a viscous mixer, or a filter screwed on to the mixer/dispenser unit or fitted within the mixer/dispenser unit. The treated or filtered material can be collected in large format containers or multiple smaller containers. Alternatively, the material can be stored in storage bottles, large transfer syringes or filling cartridges.

FIG. 12 (II) shows an exemplary medium scale operation. In a medium scale operation, the incoming material (or the material to be treated) may be in bulk quantities as well. The material can be treated in an intermediate transfer container such as filling lines, large syringes, filling cartridges. A large batch of material can be treated in the intermediate container, wherein the filter could be a mesh screen, filter tip placed within or a luer lock attachment fixed on the intermediate container. The treated or filtered material can be collected in large format containers or multiple smaller containers. Alternatively, the material can be filled in final transfer syringes for injection/direct administration to the patients.

FIG. 12 (III) shows an exemplary small scale operation. In a small scale operation, the material to be filtered//treated is filled in final transfer syringes for direct injection into the patient, for example, 1 cc syringes. The filter/sieve, for example a mesh filter, can be placed inside (or screwed onto the outlet of) the final syringe for injection into the patient. For example, a mesh screen luer lock attachment between the syringe and the needle for injection into the patient. In such instances, the treated/filtered material is directly administered to the body of the patient and does not involve ant storage or collection vessel.

Each method has different applications or advantages. For materials that recombine and readily reform aggregates, a small scale treatment immediately prior to injecting the patient would be well suited. In contrast, a large or medium scale operation, would be more appropriate for less tacky materials. These operations are higher throughput and involve a lower material cost as compared to the small scale method.

As would be appreciated by a person skilled in the art, one or more illustrative embodiments have been described by way of example. It will be understood to persons skilled in the art that a number of variations and modifications can be made without departing from the scope of the invention as defined in the claims.

What is claimed is:

1. A method of removing agglomerations of individual particles from a viscous material for administration to a patient comprising:
filling the viscous material in an extrusion system comprising at least one filter/sieve;
passing the viscous material through the at least one filter/sieve causing removal of the agglomerations of individual particles from the viscous material; wherein the filtered viscous material is suitable for administration to the patient.

2. The method of claim 1, wherein the method does not change the characteristic features or morphology of at least 98%, at least 95%, at least 92%, at least 89%, at least 86%, at least 83%, or at least 80% of the individual particles of the viscous material.

3. The method of claim 1, wherein the passing step additionally comprises forcing the viscous material through the at least one filter/sieve under pressure/by application of pressure.

4. The method of claim 3, wherein the viscous material is forced through the at least one filter/sieve by application of pressure using a plunger, an extruder, a manual compression, a syringe pump, a platen compression, a roller for flexible tubes, a compressor, or a device capable of employing similar mechanism.

5. The method of claim 1, wherein the agglomerations are removed from the viscous material by breaking-up/disintegrating the agglomerations.

6. The method of claim 1, wherein the agglomerations are removed from the viscous material by filtering out/sieving out the agglomerations that are unable to pass through the at least one filter/sieve.

7. The method of claim 1, wherein the viscous material is pre-filtered through a plurality of pre-filtering devices, wherein the pre-filtration step occurs prior to the passing step.

8. The method of claim 7, wherein the viscous material is pre-filtered through a first pre-filtering device followed by a second pre-filtering device.

9. The method of claim 7, wherein the plurality of pre-filtering devices are a series of injection devices, injection syringes, discharge systems, injection devices with luer-lock connectors, compressible tubes, non-compressible tubes, cylinders, or large-scale syringes.

10. The method of claim 8, wherein the first and second pre-filtering devices are a series of injection devices, injection syringes, or large-scale syringes, wherein the first pre-filtering device has an aperture size greater than the second pre-filtering device.

11. The method of claim 8, wherein the aperture size of the first pre-filtering device is in the range of 18G-34G.

12. The method of claim 8, wherein the aperture size of the second pre-filtering device is in the range of 18G-34G.

13. The method of claim 1, wherein the viscous material is passed through the at least one filter/sieve having a pore size/an aperture size smaller than the individual particle size of the viscous material.

14. The method of claim 13, wherein the pore size of the at least one filter/sieve is in the range of 1-1000 μm and the individual particle size is in the range of 20-1000 μm.

15. The method of claim 13, wherein the pore size of the at least one filter/sieve is in the range of 25-500 μm and the individual particle size is in the range of 40-500 μm.

16. The method of claim 13, wherein the pore size of the at least one filter/sieve is in the range of 50-200 μm and the individual particle size is in the range of 75-200 μm.

17. The method of claim 1, wherein the filtered viscous material is collected in a loading vessel/transfer syringe.

18. The method of claim 17, wherein the filtered viscous material does not occlude the loading vessel/transfer syringe when injected out of the loading vessel/transfer syringe.

19. The method of claim 18, wherein the filtered viscous material has a substantially uniform or controlled extrusion profile when injected out of the loading vessel/transfer syringe.

20. The method of claim 19, wherein the filtered viscous material injects out of the loading vessel/transfer syringe without any pressure build-ups after the initial characteristic burst/break force.

21. The method of claim 1, wherein the individual particles of the viscous material have an undamaged morphology.

22. The method of claim 1, wherein the filtered viscous material triggers an expected immune response in the patient upon administration.

23. The method of claim 1, wherein the extrusion system is selected from the group consisting of an injection syringe, a discharge system, a luer-lock connector device, or a compressible tube.

24. The method of claim 23, wherein the extrusion system is a syringe, wherein the syringe has a needle size in the range of 18G-34G.

25. The method of claim 1, wherein the loading vessel/transfer syringe is selected from the group consisting of an injection syringe, a discharge system, a luer-lock connector device, or a compressible tube.

26. The method of claim 25, wherein the loading vessel/transfer syringe is a syringe, wherein the syringe has a needle size of 18G-34G.

27. The method of any one of claims 17-26, wherein the volume of the extrusion system is greater than the volume of the loading vessel/transfer syringe, or wherein the extrusion system has a lower cross section compared to the loading vessel/transfer syringe.

28. The method of any one of claims 1-27, wherein the at least one filter/sieve is selected from the group consisting of nylon mesh, stainless steel mesh, polytetrafluoroethylene mesh or nitrocellulose mesh.

29. The method of any one of claims 1-27, wherein the at least one filter/sieve is placed inside, outside, adjacent to, or screwed on to the extrusion system.

30. The method of claim 1, wherein the at least one filter/sieve is replaced with an inline filter, a gated impeller, a static mixer, a high shear mixer, a viscous mixer or a sieving channel.

31. The method of any one of claims 1-30, wherein the passing step sizes the individual particles of the viscous material to create a substantially uniform or controlled particulate matrix for administration.

32. The method of claim 1, wherein the extrusion system is a luer-lock connector device, wherein the at least one filter/sieve is fitted/disposed within the luer-lock connector portion of the device.

33. The method of claim 32, wherein the viscous material is forced through the at least one filter/sieve fitted/disposed within the luer-lock connector portion under pressure/by application of pressure.

34. The method of claim 33, wherein the pressure is applied using a plunger, an extruder, a manual compression, a syringe pump, a platen compression, a roller for flexible tubes, a compressor or a device capable of employing similar mechanism.

35. The method of claim 33, wherein the pressure is applied using a plunger or an extruder.

36. The method of claim 1, wherein the passing step is repeated with a plurality of filters/sieves.

37. The method of claim 36, wherein the plurality of filters/sieves are of the same size or have varying sizes.

38. The method of claim 36, wherein the pore size of the at least one filter/sieve is in the range of 1-1000 μm.

39. The method of any one of claims 36-38, wherein passing the material through a plurality of filter/sieves reduces the viscosity or adjusts the particle size distribution of the viscous material to a desired level, preferably in the range of 50-750 μm.

40. The method of claim 31, wherein the method reduces the extrusion force required for injecting out the viscous material from the extrusion system.

41. The method of claim 1, wherein the viscous material is selected from the group consisting of dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

42. A method of removing agglomerations of individual particles from a viscous dermal filler material for administration to a patient comprising:
filling the dermal filler material in an extrusion system comprising at least one filter/sieve;
passing the dermal filler material through the al least one filter/sieve causing removal of the agglomerations of individual particles from the dermal filler material; wherein the filtered dermal filler material is suitable for administration to the patient.

43. The method of claim 42, wherein the method does not change the characteristic features or morphology of at least 98%, at least 95%, at least 92%, at least 89%, at least 86%, at least 83%, or at least 80% of the individual particles of the dermal filler material.

44. The method of claim 42, wherein the passing step additionally comprises forcing the dermal filler material through the at least one filter/sieve under pressure/by application of pressure.

45. The method of claim 44, wherein the dermal filler material is forced through the at least one filter/sieve by application of pressure using a plunger, an extruder, a manual compression, a syringe pump, a platen compression, a roller for flexible tubes, a compressor or a device capable of employing similar mechanism.

46. The method of claim 42, wherein the agglomerations are removed from the dermal filler material by breaking-up/disintegrating the agglomerations.

47. The method of claim 42, wherein the agglomerations are removed from the dermal filler material by filtering out/sieving out the agglomerations that are unable to pass through the at least one filter/sieve.

48. The method of claim 42, wherein the dermal filler material is pre-filtered through a plurality of pre-filtering devices, wherein the pre-filtration step occurs prior to the passing step.

49. The method of claim 48, wherein the viscous material is pre-filtered through a first pre-filtering device followed by a second pre-filtering device.

50. The method of claim 48, wherein the plurality of pre-filtering devices are selected from the group of a series of injection devices, injection syringes, discharge systems, injection devices with luer-lock connectors, compressible tubes, non-compressible tubes, cylinders, or large-scale syringes.

51. The method of claim 49, wherein the plurality of pre-filtering devices are a series of injection devices, injection syringes, or large-scale syringes, wherein the first pre-filtering device has an aperture size greater than the second pre-filtering device.

52. The method of claim 51, wherein the aperture size of the first pre-filtering device is in the range of 18G-34G.

53. The method of claim 51, wherein the aperture size of the second pre-filtering device is in the range of 18-34G.

54. The method of claim 42, wherein the dermal filler material is passed through the at least one filter/sieve having a pore size/an aperture size smaller than the individual particle size of the viscous material.

55. The method of claim 42, wherein the pore size of the at least one filter/sieve is in the range of 1-1000 μm and the individual particle size is in the range of 20-1000 μm.

56. The method of claim 42, wherein the pore size of the at least one filter/sieve is in the range of 25-500 μm and the individual particle size is in the range of 40-500 μm.

57. The method of claim 42, wherein the pore size of the at least one filter/sieve is in the range of 50-200 μm and the individual particle size is in the range of 75-200 μm.

58. The method of claim 42, wherein the filtered dermal filler material is collected in a loading vessel/transfer syringe.

59. The method of claim 58, wherein the filtered dermal filler material does not occlude the loading vessel/transfer syringe when injected out of the loading vessel/transfer syringe.

60. The method of claim 59, wherein the filtered dermal filler material has a substantially uniform or controlled extrusion profile when injected out of the loading vessel/transfer syringe.

61. The method of claim 60, wherein the filtered dermal filler material injects out of the loading vessel/transfer syringe without any pressure build-ups after the initial characteristic burst/break force.

62. The method of claim 42, wherein the individual particles of the dermal filler material have an undamaged morphology.

63. The method of claim 42, wherein the filtered dermal filler material triggers an expected immune response in the patient upon administration.

64. The method of claim 42, wherein the extrusion system is selected from the group consisting of an injection syringe, a discharge system, a luer-lock connector device, or a compressible tube.

65. The method of claim 64, wherein the extrusion system is a syringe, wherein the syringe has a needle size in the range of 18G-34G.

66. The method of claim 42, wherein the loading vessel/transfer syringe is selected from the group consisting of an injection syringe, a discharge system, a luer-lock connector device, or a compressible tube.

67. The method of claim 66, wherein the loading vessel/transfer syringe is a syringe, wherein the syringe has a needle size of 18G-34G.

68. The method of any one of claims 58-67, wherein the volume of the extrusion system is greater than the volume of the loading vessel/transfer syringe, or wherein the extrusion system has a lower cross section compared to the loading vessel/transfer syringe.

69. The method of any one of claims 42-68, wherein the at least one filter/sieve is selected from the group consisting of nylon mesh, stainless steel mesh, polytetrafluoroethylene mesh or nitrocellulose mesh.

70. The method of any one of claims 42-69, wherein the at least one filter/sieve is placed inside, outside, adjacent to, or screwed on to the extrusion system.

71. The method of claim 42, wherein the at least one filter/sieve is replaced with an inline filter, a gated impeller, a static mixer, a high shear mixer, a viscous mixer or a sieving channel.

72. The method of any one of claims 42-71, wherein the passing step sizes the individual particles of the dermal filler material to create a substantially uniform or controlled particulate matrix for administration.

73. The method of claim 42, wherein the extrusion system is a luer-lock connector device, wherein the at least one filter/sieve is fitted/disposed within the luer-lock connector portion of the device.

74. The method of claim 73, wherein the dermal filler material is forced through the at least one filter/sieve fitted/disposed within the luer-lock connector portion under pressure/by application of pressure.

75. The method of claim 74, wherein the pressure is applied using a plunger, an extruder, a manual compression, a syringe pump, a platen compression, a roller for flexible tubes, a compressor or a device capable of employing similar mechanism.

76. The method of claim 74, wherein the pressure is applied using a plunger or an extruder.

77. The method of claim 42, wherein the passing step is repeated with a plurality of filters/sieves.

78. The method of claim 77, wherein the plurality of filters/sieves are of the same size or have varying sizes.

79. The method of claim 79, wherein the pore size of the at least one filter/sieve is in the range of 1-1000 μm.

80. The method of any one of claims 77-79, wherein passing the material through a plurality of filter/sieves reduces the viscosity or adjusts the particle size distribution of the dermal filler material to a desired level, preferably in the range of 50-750 μm.

81. The method of claim 80, wherein the method reduces the extrusion force required for injecting out the dermal filler material from the extrusion system.

82. The method of claim 35, wherein the dermal filler material is selected from the group consisting of sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

83. A method of removing agglomerations of individual particles from a viscous material for administration to a patient comprising:
filling the viscous material in a first collection vessel comprising at least one filter/sieve;
passing the viscous material through the at least one filter/sieve causing removal of the agglomerations of individual particles from the viscous material
collecting the viscous material in a second collection vessel, wherein the filtered viscous material collected in the second collection vessel is suitable for administration to the patient.

84. The method of claim 83, wherein the first collection vessel comprises an extrusion system, wherein the extrusion system comprises the at least one filter/sieve.

85. The method of claim 83, wherein the method does not change the characteristic features or morphology of at least 98%, at least 95%, at least 92%, at least 89%, at least 86%, at least 83%, or at least 80% of the individual particles of the viscous material.

86. The method of claim 83, wherein the passing step additionally comprises forcing the viscous material through the at least one filter/sieve under pressure/by application of pressure.

87. The method of claim 70, wherein the viscous material is forced through the at least one filter/sieve by application of pressure using a plunger, an extruder, a manual compression, a syringe pump, a platen compression, a roller for flexible tubes, a compressor or a device capable of employing similar mechanism.

88. The method of claim 83, wherein the agglomerations are removed from the viscous material by breaking-up/disintegrating the agglomerations.

89. The method of claim 83, wherein the agglomerations are removed from the viscous material by filtering out/sieving out the agglomerations that are unable to pass through the at least one filter/sieve.

90. The method of claim 83, wherein the viscous material is pre-filtered through a plurality of pre-filtering devices, wherein the pre-filtration step occurs prior to the passing step.

91. The method of claim 90, wherein the viscous material is pre-filtered through a first pre-filtering device followed by a second pre-filtering device.

92. The method of claim 83, wherein the plurality of pre-filtering devices are selected from the group of a series of injection devices, injection syringes, discharge systems, injection devices with luer-lock connectors, compressible tubes, non-compressible tubes, cylinder, or large-scale syringes.

93. The method of claim 91, wherein the plurality of pre-filtering devices are a series of injection devices, injection syringes, or large-scale syringes, wherein the first pre-filtering device has an aperture size greater than the second pre-filtering device.

94. The method of claim 91, wherein the aperture size of the first pre-filtering device is selected from 18G-34G.

95. The method of claim 91, wherein the aperture size of the second pre-filtering device is selected from 18G-34G.

96. The method of claim 83, wherein the viscous material is passed through the at least one filter/sieve having a pore size/an aperture size smaller than the individual particle size of the viscous material.

97. The method of claim 83, wherein the pore size of the at least one filter/sieve is in the range of 1-1000 μm and the individual particle size is in the range of 20-1000 μm.

98. The method of claim 83, wherein the pore size of the at least one filter/sieve is in the range of 25-500 μm and the individual particle size is in the range of 40-500 μm.

99. The method of claim 83, wherein the pore size of the at least one filter/sieve is in the range of 50-200 μm and the individual particle size is in the range of 75-200 μm.

100. The method of claim 83, wherein the filtered viscous material collected in the second collection vessel is dispensed/injected out using a dispensing device.

101. The method of claim 100, wherein the filtered viscous material does not occlude the dispensing device when dispensed/injected out of the dispensing device.

102. The method of claim 100, wherein the filtered viscous material has a substantially uniform or controlled extrusion profile when dispensed/injected out of the dispensing device.

103. The method of claim 102, wherein the filtered viscous material dispenses/injects out of the dispensing device without any pressure build-ups after the initial characteristic burst/break force.

104. The method of claim 103, wherein the individual particles of the viscous material have an undamaged morphology.

105. The method of claim 83, wherein the filtered viscous material triggers an expected immune response in the patient upon administration.

106. The method of claim 100, wherein the dispensing device is selected from the group consisting of a series of injection devices, injection syringes, discharge systems, injection devices with luer-lock connectors, compressible tubes, non-compressible tubes, cylinders, or large-scale syringes.

107. The method of claim 83, wherein the dispensing device is a syringe, wherein the syringe has a needle size of 18G-34G.

108. The method of claim 83, wherein the at least one filter/sieve is selected from the group consisting of nylon mesh, stainless steel mesh, polytetrafluoroethylene mesh or nitrocellulose mesh.

109. The method of claim 83, wherein the at least one filter/sieve is placed inside, outside, adjacent to, or screwed on to the first collection vessel.

110. The method of claim 83, wherein the at least one filter/sieve is replaced with an inline filter, a gated impeller, a static mixer, a high shear mixer, a viscous mixer or a sieving channel.

111. The method of claim 83, wherein the passing step sizes the individual particles of the viscous material to create a substantially uniform or controlled particulate matrix for administration.

112. The method of claim 83, wherein the at least one filter/sieve is disposed within a luer-lock connector fitted within the first collection vessel/a luer-lock connector portion of the first collection vessel.

113. The method of claim 112, wherein the viscous material is forced through the at least one filter/sieve disposed within the luer-lock connector portion under pressure/by application of pressure.

114. The method of claim 113, wherein the pressure is applied using a plunger, an extruder, a manual compression, a syringe pump, a platen compression, a roller for flexible tubes, a compressor or a device capable of employing similar mechanism.

115. The method of claim 113, wherein the pressure is applied using a plunger or an extruder.

116. The method of claim 83, wherein the passing step is repeated with a plurality of filters/sieves.

117. The method of claim 116, wherein the plurality of filters/sieves are of the same size or have varying sizes.

118. The method of claim 116, wherein the size of the at least one filter/sieve is in the range of 1-1000 μm.

119. The method of claim 116, wherein passing the material through a plurality of filter/sieves reduces the viscosity or adjusts the particle size distribution of the viscous material to a desired level, preferably in the range of 50-750 μm.

120. The method of claim 119, wherein the method reduces the extrusion force required for injecting out the viscous material from the extrusion system.

121. The method of claim 83, wherein the viscous material is selected from the group consisting of dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

122. The method of claim 83, wherein the first collection vessel is a series of vessels of varying dimensions, a storage unit or chamber, an industrial mixer, an industrial dispenser, an intermediate transfer container, a injection syringe, a large-injection syringe, a luer-lock connector device, a discharge system, or a compressible tube.

123. The method of claim 83, wherein the second collection vessel is selected from at least one of a transfer container, a storage bottle, a injection syringe, a filler cartridge, a series of vessels of varying dimensions, a storage unit or chamber, an industrial mixer, an industrial dispenser, an intermediate transfer container, a large-injection syringe, a luer-lock connector device, a discharge system, or a compressible tube.

124. The method of claim 123, wherein the second collection vessel is used for dispensing/injecting out the filtered viscous material for administration to the patient.

125. The method of claim 83, wherein the first collection vessel has a volume greater than the second collection vessel.

126. Use of the method of any one of the claim 1-41 or 83-125 to reduce the viscosity or adjust the particle size distribution of a viscous material to a desired level, preferably in the range of 50-750 μm.

127. Use of the method of claim 42-82 to reduce the viscosity or adjust the particle size distribution of a dermal filler material to a desired level, preferably in the range of 50-750 μm.

128. Use of the method of any of the claim 1-41 or 83-125 to break down agglomerates/aggregates in a viscous material.

129. Use of the method of claim 42-82 to break down agglomerates/aggregates in a dermal filler material.

130. Use of the method of any one of the claim 1-41 or 83-125 to prevent needle occlusions in an injection or dispensing device during delivery of a viscous material.

131. Use of the method of claim 42-82 to prevent needle occlusions in an injection or dispensing device during delivery of a dermal filler material.

132. Use of the method of any one of the claim 1-41 or 83-125 to size the individual particles of a viscous material such that the viscous material has a nearly/substantially uniform individual particle size.

133. Use of the method of claim 42-82 to size the individual particles of a dermal filler material such that the dermal filler material has a nearly/substantially uniform individual particle size.

134. Use of the method of any one of the claim 1-41 or 83-125 to reduce the individual particle size of a viscous material without affecting/impacting its rheological properties.

135. Use of the method of any one of the claim 1-41 or 83-125 to reduce the individual particle size of a viscous material without affecting/impacting/changing the characteristic features or morphology of the individual particles of the viscous material.

136. Use of the method of claim 42-82 to reduce the individual particle size of a viscous material without affecting/impacting its rheological properties.

137. Use of the method of any one of the claims 42-82 to reduce the individual particle size of a viscous material without affecting/impacting/changing the characteristic features or morphology of the individual particles of the viscous material.

138. Use of the method of any one of the claim 1-41 or 83-125 to filter a viscous material such that the viscous material has a uniform or controlled particulate matrix for administration.

139. Use of the method of claim 42-82 to filter a dermal filler material such that the dermal filler material has a uniform or controlled particulate matrix for administration.

140. Use of the method of any one of the claim 1-41 or 83-125 wherein the viscous material is selected from the group of dermal fillers; sealants; adhesives; composite mixtures of mammalian cells; scaffolding materials; bone pastes; bone cements; cartilage biomaterials; injectables including venous stasis applications; protein hydrogel; carbohydrate hydrogels including cellulose, pectin, and lignin; cell material mixtures; thickeners; gelling agents; and stabilizers.

141. Use of the method of any one of the claim 1-41 or 83-125 to reduce the individual particle size of the viscous material thereby reducing the extrusion force required for injecting out the viscous material from the extrusion system.

142. Use of the method of any one of the claims 42-82 to reduce the individual particle size of the dermal filler material thereby reducing the extrusion force required for injecting out the dermal filler material from the extrusion system.

143. A device for removing agglomerations of individual particles from a viscous material comprising:
an extrusion system; and
at least one filter/sieve placed inside, outside, adjacent to, or screwed on to the extrusion system;
wherein passing the viscous material through the at least one filter/sieve causes removal of the agglomerations of individual particles from the viscous material.

144. The device of claim 143, additionally comprising an extruder to force the viscous material through the at least one filter/sieve under pressure/by application of pressure.

145. The device of claim 144, wherein the extruder pressure is regulated by electronic means.

146. The device of claim 142, wherein the at least one filter/sieve is selected from the group consisting of nylon, stainless steel, polytetrafluoroethylene, or nitrocellulose.

147. The device of claim 142, wherein the at least one filter/sieve is a nylon mesh or a stainless steel mesh.

148. The device of claim 142, wherein the at least one filter/sieve is replaced with an inline filter, a gated impeller, a static mixer, a high shear mixer, a viscous mixer or a sieving channel.

149. The device of claim 148, wherein the dimensions of the inline filter or sieving channel are in the range of 0.5 mm to 2540 mm.

150. The device of claim 142, wherein the extrusion system is connected to a plurality of pre-filtering devices for pre-filtering the viscous material before passing the material through the at least one filter/sieve.

151. The device of claim 150, wherein the extrusion system is connected to at least one pre-filtering device and a loading vessel/transfer syringe, wherein the at least one pre-filtering device is connected to the loading vessel/transfer syringe.

152. The device of claim 142, wherein the extrusion system is coupled to the loading vessel/transfer syringe for collecting the filtered viscous material.

153. The device of claim 142, wherein the at least one filter/sieve has an aperture size smaller than the individual particle size of the viscous material.

154. The device of claim 142, wherein the at least one filter/sieve has an aperture size ranging from 1-1000 μm and the individual particle size is in the range of 20-1000 μm.

155. The device of claim 142, wherein the at least one filter/sieve has an aperture size ranging from 25-500 μm and the individual particle size is in the range of 40-500 μm.

156. The device of claim 142, wherein the at least one filter/sieve has an aperture size ranging from 50-200 μm and the individual particle size is in the range of 75-200 μm.

157. The device of claim 142, wherein the extrusion system is selected from the group of an injection syringe, a discharge system, a luer-lock connector device, or a compressible tube.

158. The device of claim 142, wherein the extrusion system is a syringe.

159. The device of claim 158, wherein the syringe has a needle size of 18G-34G.

160. The device of claim 142, wherein the loading vessel/transfer syringe is selected from the group of an injection syringe, a discharge system, a luer-lock connector device, or a compressible tube.

161. The device of claim 142, wherein the loading vessel/transfer syringe is a syringe.

162. The device of claim 161, wherein the syringe has a needle size of 18G-34G.

163. The device of claim 142, wherein the extrusion system has a volume greater than the volume of the loading vessel/transfer syringe or wherein the extrusion system has a lower cross section compared to the loading vessel/transfer syringe.

164. The device of claim 142, wherein the extrusion system is a luer-lock connector injection device.

165. The device of claim 164, wherein the at least one filter/sieve is disposed within the luer-lock connector.

166. A device for removing agglomerations of individual particles from a viscous material comprising:
a first collection vessel;
at least one filter/sieve fitted inside, outside, adjacent to, or screwed on to the first collection vessel, wherein passing the viscous material through the at least one filter/sieve causes removal of the agglomerations of individual particles from the viscous material; and
a second collection vessel coupled to the first collection vessel for collecting the filtered viscous material.

167. The device of claim 166, wherein the first collection vessel comprises the extrusion system as defined in claims 143-165.

168. The device of claim 166, wherein the first collection vessel is selected from the group of a series of vessels with varying dimensions, a storage unit or chamber, an industrial mixer, an industrial dispenser, an intermediate transfer container, a injection syringe, a large-injection syringe, a luer-lock connector device, a discharge system, or a compressible tube.

169. The device of claim 166, wherein the second collection vessel is selected from the group of a transfer container, a storage bottle, a injection syringe, a filler cartridge, a series of vessels with varying dimensions, a storage unit or chamber, an industrial mixer, an industrial dispenser, an intermediate transfer container, a large-injection syringe, a luer-lock connector device, a discharge system, or a compressible tube.

170. The device of claim 166, wherein an additional filter/sieve is fitted within the second collection vessel.

171. The device of claim 166, wherein the second collection vessel is a collection chamber disposed within the first collection vessel.

172. The device of claim 166, wherein the device additionally comprises an extruder to force the viscous material through the at least one filter/sieve under pressure/by application of pressure.

173. The device of claim 1, wherein the extruder pressure is regulated by electronic means.

174. The device of claim 166, wherein the at least one filter/sieve has an aperture size ranging from 1-1000 μm and the individual particle size is in the range of 20-1000 μm.

175. The device of claim 166, wherein the at least one filter/sieve has an aperture size ranging from 25-500 μm and the individual particle size is in the range of 40-500 μm.

176. The device of claim 166, wherein the at least one filter/sieve has an aperture size ranging from 50-200 μm and the individual particle size is in the range of 75-200 μm.

177. The device of claim 167, wherein the at least one filter/sieve is selected from a nylon mesh, a stainless steel mesh, a polytetrafluoroethylene mesh, or a nitrocellulose mesh.

178. The device of claim 166, wherein the at least one filter/sieve is replaced with an inline filter, a gated impeller, a static mixer, a high shear mixer, a viscous mixer or a sieving channel.

179. The device of claim 166, wherein the first collection vessel is connected to a plurality of pre-filtering devices for pre-filtering the viscous material before passing the material through the at least one filter/sieve.

180. The device of claim 166, wherein the first collection vessel is connected to a plurality of pre-filtering device, and the pre-filtering devices are connected to the second collection vessel.

181. The device of claim 166, wherein the first collection vessel is connected to a first pre-filtering device and a second pre-filtering device, and the pre-filtering devices are connected to the second collection vessel.

182. The device of claim 166, wherein the at least one filter/sieve has an aperture size smaller than the individual particle size of the viscous material.

183. The device of claim 166, wherein a luer-lock connector is fitted within the first collection vessel.

184. The device of claim 183, wherein the at least one filter/sieve is placed inside the luer-lock connector fitted within the first collection vessel.
